(19) United States
(12) Patent Application Publication
(10) Pub. No.: US 2026/0114454 A1
(43) Pub. Date: Apr. 30, 2026

(54) INSECTICIDAL PSEUDOMONAS SP. 14OG COMPOSITIONS AND USES THEREOF

(71) Applicant: St. Gabriel Organics, Orange, VA (US)

(72) Inventor: Benjamin Wellborn, Orange, VA (US)

(73) Assignee: St. Gabriel Organics, Orange, VA (US)

(21) Appl. No.: 19/221,455

(22) Filed: May 28, 2025

Related U.S. Application Data
(60) Provisional application No. 63/653,109, filed on May 29, 2024.

(51) Int. Cl. A01N 63/27; A01N 63/50; A01P 7/04; C07K 14/21; C12N 1/205; C12R 1/38

(52) U.S. Cl. CPC A01N 63/27; A01N 63/50; A01P 7/04; C07K 14/21; C12N 1/205; C12R 2001/38

(57) ABSTRACT

The present invention relates to a novel bacterium Pseudomonas sp. 14og as deposited into the NRRL Patent Culture Collection on 3 Dec. 2020 and designated NRRL accession no. B-67999. The present invention also relates to insecticidally active substances obtained or derived from Pseudomonas sp. 14og. The insecticidal substances find use in controlling, inhibiting growth or killing Lepidopteran, Coleopteran, Dipteran, Hemipteran, Tetranychidae, fungal, nematode and mite pest populations and for producing compositions with insecticidal activity. The present invention relates to a novel bacterium and use for biocontrol of insect pests. More particularly, the invention relates to a newly discovered species of Pseudomonas bacterium that exhibits insecticidal activity. The invention also relates to proteins and/or peptides and/or metabolites obtained from the strain that possess insecticidal activity and to insecticidal compositions comprising cultures of the novel strain and/or supernatants, filtrates, and extracts obtained from the strain, and use thereof to control insect pests. The present invention relates to a novel bacterium and uses thereof. In particular, the present invention relates to applications concerned with the biopesticidal activity of the novel bacterium and of its mutant or variant strains. The present invention also concerns proteinaceous substances produced by this novel bacterium and of its mutant or variant strains obtained or derived from the aforesaid bacteria.

### FIELD OF THE INVENTION

This disclosure relates to the field of molecular biology. Provided are novel genes that encode a microorganism having pesticidal qualities and that produces pesticidal products. These pesticidal products and the nucleic acid sequences that encode them are useful in preparing pesticidal formulations and in the production of pesticide products and/or transgenic pest-resistant plants.

### REFERENCE TO SEQUENCE LISTING SUBMITTED ELECTRONICALLY

The official copy of the sequence listing is submitted electronically via Patent Center as an WIPO ST.26 XML formatted sequence listing with a file named “Sequence_listing_162001-000110US” created on Dec. 10, 2025, and having a size of 22,646 bytes and is filed concurrently with the specification. The sequence listing contained in this formatted document is part of the specification and is herein incorporated by reference in its entirety.

### BACKGROUND OF THE INVENTION

Insect pests are a major cause of crop losses. In the United States alone, billions of dollars are lost every year due to infestation by various genera of insects. In addition to losses in field crops, insect pests are also a burden to vegetable and fruit growers to producers of ornamental flowers, and they are a nuisance to gardeners and homeowners.

Certain species of microorganisms of the genus Bacillus are known to possess pesticidal activity against a range of insect pests including Lepidoptera, Diptera, Coleoptera, Hemiptera and others. Bacillus thuringiensis (Bt) and Bacillus popilliae are among the most successful biocontrol agents discovered to date. Insect pathogenicity has also been attributed to strains of B. larvae, B. lentimorbus, B. sphaericus and B. cereus. Microbial insecticides, particularly those obtained from Bacillus strains, have played an important role in agriculture as alternatives to chemical pest control.

The continued use of chemical pesticides over many years can lead to an increase in resistant insects. There is, therefore, a need for novel biopesticides to control insects and to provide an alternative to chemical pesticides which can be toxic to non-target organisms in the environment. There remains a need to identity new and effective biological pest control agents that provide an economic benefit to farmers and that are environmentally acceptable. Particularly needed are entomopathogenic microorganisms, proteins and/or peptides and/or biologically produced metabolites that are toxic to pestiferous insects that have a different mode of action than existing insect control products as a way to mitigate the development of resistance.

### SUMMARY OF THE INVENTION

A novel insecticidal bacterium is described. The bacterium is a species and has been named Pseudomonas sp. 14og.

We have discovered a new species of Pseudomonas bacterium which is distinct from all other described species in the genus and which exhibits insecticidal activity. The bacterium is a species residing within the Pseudomonas fluorescens complex and we have designated it as Pseudomonas sp. 14og. A biologically pure culture of Pseudomonas sp. 14og. has been deposited with the Agricultural Research Service Culture Collection (NRRL) under the provisions of the Budapest Treaty on the International Recognition of the Deposit of Microorganisms for the Purpose of Patent Procedure and has been given the accession number NRRL B-67999.

The unique strain of the invention, also denoted as Pseudomonas sp. 14og NRRL B-67999, exhibits effective insecticidal activity, and cultures of the new bacterium are useful for control of insect pests.

The unique strain of the invention produces one or more active proteins and/or peptides and/or metabolites that possess insecticidal activity. Toxicity is released into the supernatant obtained from the strain; viable bacterial cells are not required for insecticidal activity. Accordingly, the present invention is also directed to insecticidally active supernatants and filtrates obtained from the unique strain. The one or more toxic proteins and/or peptides and/or metabolites exhibit insecticidal activity, and the invention is further directed to insecticidally active extracts obtained from the strain of the invention.

The present inventors have shown that lipopeptides and/or rhamnolipids and/or proteins and/or peptides and/or metabolites produced by Pseudomonas sp. 14og are capable of killing or inhibiting the growth of scarab beetle larvae. This inhibition is due to a pesticidal activity, which is particularly advantageous in the field of turfgrass or crop treatment.

The subject of the present invention is therefore the use of Pseudomonas sp. 14og or substances obtained or derived from Pseudomonas sp. 14og for killing or inhibiting the growth of scarab beetle larvae, comprising the use of live or killed Pseudomonas sp. 14og cells or cultures, lipopeptides and/or rhamnolipid and/or proteins and/or peptides and/or metabolites produced by bacteria of Pseudomonas sp. 14og.

By “lipopeptide” is meant here generally cyclic peptides produced by bacteria such as Bacillus and Pseudomonas. They are mainly consisting of a hydrophilic peptide part, for example containing from 7 to 25 amino acids, connected to a hydrophobic lipid part, most often a fatty acid. Lipopeptides are well known to those skilled in the art because of their biological properties. For example, many of them have surfactant properties or have antibacterial or antifungal activities that have attracted the interest of manufacturers. However, this is the first time that an activity of these molecules, produced by Pseudomonas sp. 14og, against coleopteran larvae is highlighted.

By “metabolite” is meant here organic molecules having a molar mass of less than 1000, produced by bacteria such as Bacillus and Pseudomonas as an intermediate or end product of metabolism. Metabolites are well known to those skilled in the art because of their biological properties. For example, many of them have metal chelating properties or have antimicrobial activities s that have attracted the interest of manufacturers. Examples of metabolites may include siderophores, fluorescent molecules, or unique amino acids.

The invention describes a pesticidal strain of a new species to be called Pseudomonas sp. 14og residing within the Pseudomonas fluorescens complex

The present invention is further directed to methods of controlling insects using the unique bacterium of the invention. This aspect includes application of an effective insect control amount of the strain cells, supernatant, filtrate, permeate, precipitate, or extract containing an insecticidally active protein and/or peptide and/or metabolite produced by the strain or combinations thereof. Pseudomonas sp. 14og and/or a protein and/or peptide and/or metabolite obtained from NRRL B-67999 has been shown to be toxic to scarab beetle larvae.

By “permeate” is meant here organic molecules having passed through a molecular weight cutoff sieve. Permeates are well known to those skilled in the art because of their biological properties. For example, many of them have pharmaceutical properties or have biochemical activities that have attracted the interest of manufacturers. However, this is the first time that an activity of the molecules, produced by Pseudomonas sp. 14og, against coleopteran larvae is highlighted.

By “precipitate” is meant here organic molecules having a lower solubility in water in the presence of ammonium sulfate, similar salts, or alcohols. Precipitates are well known to those skilled in the art because of their biological properties. For example, many of them have enzymatic properties or have pesticidal activities that have attracted the interest of manufacturers. However, this is the first time that an activity of these molecules, produced by Pseudomonas sp. 14og, against coleopteran larvae is highlighted.

Pseudomonas sp. 14og is active against scarab beetle larvae. Death occurs within 30 days post inoculation. The infection process appears to be due to invasion of the haemocoel followed by a rapid build up in the bacterial population leading to the cadaver taking on an amber or black appearance. Data are provided on biochemical utilization tests (Biolog). DNA sequences relating to phylogenetic analysis encompassing 16s ribosomal RNA sequencing and MLST sequence analysis of known Pseudomonas genes are given.

In addition the novel bacterium also produces a toxic permeate component that is also useful as a biopesticide agent in the control of scarab beetle larvae. Larvae immersed in a solution of permeate passed through a 5 kilodalton tangential flow filter die within 30 days post inoculation.

In addition the novel bacterium also produces a toxic, precipitated component that is also useful as a biopesticide agent in the control of scarab beetle larvae. Larvae immersed in a solution of precipitated material produced by ammonium sulfate precipitation die within 30 days post inoculation.

In one aspect, described below is a method of inhibiting growth of an insect that includes contacting the insect with dried whole broth culture of Pseudomonas 14og.

In another aspect, described below is a method of inhibiting growth of an insect that includes contacting the insect with an isolated permeate.

In one embodiment, the insect is selected from the group consisting of Coleopteran, Lepidopteran, Dipteran, or Hemipteran insects.

In another aspect, the insect is a herbivorous insect or a pesticide resistant insect.

In another aspect, described herein is a method of inhibiting growth of an insect that includes contacting the insect with a composition containing a Pseudomonas 14og strain, a Pseudomonas 14og cell lysate, a Pseudomonas 14og polypeptide, or a Pseudomonas 14og culture filtrate. The insect is a Coleopteran species. In one embodiment, the insect is of the genus Popilliae, Anomala, Cyclocephala, Melolontha, Amphimallon or Phyllophaga. In one embodiment, the culture filtrate is a water soluble molecule. The Pseudomonas 14og strain can be cultured in a minimal salts medium and the culture filtrate can be obtained by numerous methods known in the state of the art. In another embodiment Pseudomonas 14og strain can be cultured in a super optimal broth and the culture precipitate can be obtained by numerous methods known in the state of the art. In another embodiment a whole broth culture of the Pseudomonas 14og strain can be dried by numerous methods known in the state of the art. In another embodiment a whole culture of the Pseudomonas 14og strain can be grown on solid state media and dried by numerous methods known in the state of the art. In another embodiment a whole culture of the Pseudomonas 14og strain can be grown on solid state media and active molecules produced by Pseudomonas sp. 14og can be collected by washing the cells and/or proteins and/or peptides and/or metabolites off of the media with water alone or in combination with detergents and, or solvents and, or agitation and, or numerous other methods known in the state of the art.

In another aspect, the present invention provides methods of inhibiting the growth of an insect. In one embodiment, the methods comprise contacting the insect with an effective amount of a product described herein. In other embodiments, the methods comprise contacting the insect with a genetically similar organism comprising a similar products described herein or a product produced by the similar organism.

A further aspect of the invention pertains to compositions which incorporate the strain of the invention and/or compositions comprising an insecticidally active culture and/or protein and/or peptide and/or metabolite produced by the strain of the invention. Such compositions include, for example, whole cultures or suspensions of the strain; supernatants, filtrates or extracts obtained from the strain or combinations of the foregoing. Such compositions may optionally include other ingredients such as an agricultural carrier, insect feeding stimulant, spreading agent, sticking agent, thickener, emulsifier, stabilizer, preservative, pheromones, other attractants, fungicides, other insecticidal toxicants including other microbes and/or their proteins and/or peptides and/or metabolites, buffer, water, diluent or other additives as known in the art of formulation of insecticidal compositions. The bacterial biocontrol agent of the present invention may also be used in combination with chemical compounds, including for example feeding stimulants, photoactive dyes, fluorescent brighteners (also denoted as optical brighteners), fungicides, and other insecticides. Photoactive dyes in combination with biological control agents are described by Martin et al., 1998. Fluorescent brighteners, particularly those which provide protection for pathogens from the damaging effects of exposure to UV radiation and which may enhance biological activity of the organism, are described in U.S. Pat. No. 5,124,149 to Shapiro et al. The compositions are applied as known in the art to protect plants from insect pests, including for example, application to soil in a field or surrounding a plant, to target plants, e.g., plant roots, on plant foliage, stems, flowers, tubers, seedlings, and seeds. The strain of the invention can be grown on rice grains or cracked wheat, and the rice grains or cracked wheat applied to the plant or soil.

The invention provides a new control means against agriculturally important pestiferous insects such as the Japanese beetle, and gives growers alternatives to B. thuringiensis, as well as chemicals, for control of this and other pestiferous insects. Expanded use of biologicals for the control of the Japanese beetle and other insect pests will improve resistance management, reduce pesticide use, and produce novel compounds for potential use in transgenic plants. Because the new strain of the invention is a Gram-negative bacterium, it is unlikely to have mechanisms similar to B. thuringiensis for killing, and could be employed in resistance management.

In accordance with our discovery, it is an object of the invention to provide a biologically pure culture of a new species of Pseudomonas. In one aspect, the invention is directed to Pseudomonas sp. 14og and strains having the identifying characteristics of NRRL B-67999 and use thereof as biological control agents against insect pests. In another aspect, the invention is directed to Pseudomonas strains which have a 16S rDNA gene sequence of SEQ ID NO: 1.

A further object of the invention is the provision of methods for biologically controlling insect pests using the strain of the invention, an insecticidally-active protein and/or peptide and/or metabolite obtained from the strain, and agricultural compositions which incorporate the strain or an insecticidally-active protein and/or peptide and/or metabolite obtained from the strain.

Other objects and advantages of the invention will become readily apparent from the ensuing drawings and description.

### BRIEF DESCRIPTION OF DRAWINGS

Further aspects of the present invention will become apparent from the following description which is given by way of example only and with reference to the accompanying drawings in which:

FIG. 1 shows a pairwise comparison of the six total 16s ribosomal DNA sequences of Pseudomonas sp. 14og within the genome, using the program BLAST® pairwise alignments, showing that three of the total 16s ribosomal DNA sequences are identical and the three other 16s ribosomal DNA sequences each have two unique substitutions;

FIG. 2 shows a phylogenetic comparison based on the 16s ribosomal DNA sequence (identified in FIG. 1 as having three identical copies within the Pseudomonas sp. 14og genome) using the program BLAST® pairwise alignments. This tree was produced using BLAST® pairwise alignments and the Neighbor-Joining method;

FIG. 3 shows a phylogenetic comparison based on data from glnS. This tree was produced using BLAST® pairwise alignments and the Neighbor-Joining method;

FIG. 4 shows a phylogenetic comparison based on data from gyrB, This tree was produced using BLAST® pairwise alignments and the Neighbor-Joining method;

FIG. 5 shows a phylogenetic comparison based on data from ileS, This tree was produced using BLAST® pairwise alignments and the Neighbor-Joining method;

FIG. 6 shows a phylogenetic comparison based on data from nuoD, This tree was produced using BLAST® pairwise alignments and the Neighbor-Joining method;

FIG. 7 shows a phylogenetic comparison based on data from recA, This tree was produced using BLAST® pairwise alignments and the Neighbor-Joining method;

FIG. 8 shows a phylogenetic comparison based on data from rpoB, This tree was produced using BLAST® pairwise alignments and the Neighbor-Joining method;

FIG. 9 shows a phylogenetic comparison based on data from rpoD, This tree was produced using BLAST® pairwise alignments and the Neighbor-Joining method;

### DETAILED INVENTION

All references, including any patents or patent applications cited in this specification are hereby incorporated by reference. No admission is made that any reference constitutes prior art. The discussion of the references states what their authors assert, and the applicants reserve the right to challenge the accuracy and pertinence of the cited documents. It will be clearly understood that, although a number of prior art publications are referred to herein, this reference does not constitute an admission that any of these documents form part of the common general knowledge in the art, in the United States or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

It is an object of the present invention to address the foregoing problems or at least to provide the public with a useful choice.

Further aspects and advantages of the present invention will become apparent from the ensuing description which is given by way of example only.

The original organism name Pseudomonas sp. 14og refers to the same organism as originally described in the provisional specification and the subject of the biological deposited into the NRRL Patent Culture Collection on 3 Dec. 2020 and designated NRRL accession no. B-67999.

It should be appreciated by those skilled in the art that unless the context clearly relays otherwise use of the terms Pseudomonas sp. 14og or bacteria in this specification should also be taken to include mutant and variant strains of Pseudomonas sp. 14og which retain the biopesticide activity.

According to a first aspect of the present invention there is provided an isolated Pseudomonas sp. 14og bacterium deposited into the NRRL Patent Culture Collection on 3 Dec. 2020 and designated NRRL accession no. B-67999.

According to a second aspect of the present invention there is provided the use of Pseudomonas sp. 14og to directly or indirectly obtain a biopesticide.

According to a third aspect of the present invention there is provided the use of Pseudomonas sp. 14og as a biopesticide.

According to a fourth aspect of the present invention there is provided the use of Pseudomonas sp. 14og in the manufacture of a composition suitable as a biopesticide.

According to a fifth aspect of the present invention there is provided a culture of Pseudomonas sp. 14og bacterium deposited into the NRRL Patent Culture Collection on 3 Dec. 2020 and designated NRRL accession no. B-67999.

According to a sixth aspect of the present invention there is provided the use of a culture of Pseudomonas sp. 14og to directly or indirectly obtain a biopesticide.

According to a seventh aspect of the present invention there is provided the use of a culture of Pseudomonas sp. 14og as a biopesticide.

According to a eighth aspect of the present invention there is provided the use of a culture of Pseudomonas sp. 14og in the manufacture of a composition suitable as a biopesticide.

According to a ninth aspect of the present invention there is provided a cellular extract obtained from Pseudomonas sp. 14og bacterium deposited into the NRRL Patent Culture Collection on 3 Dec. 2020 and designated NRRL accession no. B-67999 or a culture thereof.

According to a tenth aspect of the present invention there is provided the use of a cellular extract of Pseudomonas sp. 14og to directly or indirectly obtain a biopesticide.

According to an eleventh aspect of the present invention there is provided the use of a cellular extract of Pseudomonas sp. 14og as a biopesticide.

According to a twelfth aspect of the present invention there is provided the use of a cellular extract of Pseudomonas sp. 14og in the manufacture of a composition suitable as a biopesticide.

According to a thirteenth aspect of the present invention there is provided the use of a sonicated cell filtrate of Pseudomonas sp. 14og which has a biopesticide activity.

According to a fourteenth aspect of the present invention there is provided the use of a sonicated cell filtrate of Pseudomonas sp. 14og to directly or indirectly obtain a biopesticide.

According to a fifteenth aspect of the present invention there is provided the use of a sonicated cell filtrate of Pseudomonas sp. 14og as a biopesticide.

According to a sixteenth aspect of the present invention there is provided the use of a sonicated cell filtrate of Pseudomonas sp. 14og in the manufacture of a composition suitable as a biopesticide.

According to a further aspect of the present invention there is provided a supernatant of a whole broth culture of Pseudomonas sp. 14og bacterium deposited into the NRRL Patent Culture Collection on 3 Dec. 2020 and designated NRRL accession no. B-67999.

According to a further aspect of the present invention there is provided the use of the supernatant of a whole broth culture of Pseudomonas sp. 14og as a biopesticide.

According to a further aspect of the present invention there is provided the use of the supernatant of a whole broth culture of Pseudomonas sp. 14og to directly or indirectly obtain a biopesticide.

According to a further aspect of the present invention there is provided the use of the supernatant of a whole broth culture of Pseudomonas sp. 14og in the manufacture of a composition suitable as a biopesticide.

According to a further aspect of the present invention there is provided a composition that includes an effective amount of Pseudomonas sp. 14og wherein said bacteria exhibits a biopesticide activity.

According to a further aspect of the present invention there is provided a composition formulated from an effective amount of a culture of Pseudomonas sp. 14og wherein said culture exhibits a biopesticide activity.

According to a further aspect of the present invention there is provided a composition included an effective amount Pseudomonas sp. 14og, wherein Pseudomonas sp. 14og has been killed as an intact form and maintains a biopesticide activity.

According to a further aspect of the present invention there is provided a composition formulated from an effective amount of a whole broth culture of Pseudomonas sp. 14og wherein said whole broth culture exhibits a biopesticide activity.

According to a further aspect of the present invention there is provided a composition formulated from an effective amount of a supernatant of a whole broth culture of Pseudomonas sp. 14og wherein the supernatant from the culture exhibits a biopesticide activity.

According to a further aspect of the present invention there is provided a composition formulated from an effective amount of a cellular extract of Pseudomonas sp. 14og wherein said extract exhibits a biopesticide activity.

According to a further aspect of the present invention there is provided a composition formulated from an effective amount of a sonicated cell filtrate of Pseudomonas sp. 14og, wherein said extract exhibits a biopesticide activity.

In one example, the composition may be formulated with at least one biopolymer compound. For example, at least one biopolymer compound is at least one type of gum compound.

In another example, the composition may be formulated as a gel composition.

In another example, the composition may be formulated with at least one biopolymer compound and at least one desiccating agent.

In another example, the composition may be formulated with at least one cryoprotecting compound and at least one desiccating agent.

In another example, the composition may be formulated with at least one type of gum compound and the at least one desiccating agent is at least one inert siliceous compound.

In another example, the composition may be formulated with at least one type of gum compound and the at least one desiccating agent is at least one abrasive siliceous compound.

In another example, the composition may be formulated with at least one type of carbohydrate compound and the at least one desiccating agent is at least one abrasive siliceous compound.

In another example, the composition may be formulated with at least one type of proteinaceous compound and the at least one desiccating agent is at least one abrasive siliceous compound.

In another example, the composition may be formulated with at least one type of lyoprotectant compound and the at least one desiccating agent is at least one abrasive siliceous compound.

In another example, the composition may be formulated with at least one type of gum compound and the at least one desiccating agent is at least one inert clay compound.

In another example, the composition may be formulated as a dough or granular material.

In another example, the composition may be formed into a prill or granule shape.

In another example, the composition may be mixed with an aqueous liquid and sprayed onto a substrate. Other embodiments, the composition may be coated onto a substrate. For example, the substrate may be a seed.

In another example, the composition may be mixed with or added to another microbial pesticide. In other embodiments, the composition may be added to Paenibacillus popilliae. In other embodiments, the composition may be added to Bacillus thuringiensis. In other embodiments, the composition may be added to other insecticidal microorganisms. In other embodiments, the composition may be added to other insecticidal chemicals.

According to a further aspect of the present invention there is provided a method of treating or protecting a plant and/or plant derived materials from pest infestation wherein the method comprises applying to the plant or its environment an effective amount of Pseudomonas sp. 14og. In other embodiments, the composition may be added to water. In other embodiments, the composition may be added to fertilizing materials. In other embodiments, the composition may be added to other soil amending materials.

According to a further aspect of the present invention there is provided a method substantially as described above wherein the effective amount of Pseudomonas sp. 14og is obtained from a culture of Pseudomonas sp. 14og.

According to a further aspect of the present invention there is provided a method substantially as described above wherein the effective amount of Pseudomonas sp. 14og is obtained from a supernatant from a whole broth culture of Pseudomonas sp. 14og.

According to a further aspect of the present invention there is provided a method of treating or protecting a plant or plant derived materials from pest infestation wherein the method comprises applying to the plant or its environment an effective amount of a cellular extract of Pseudomonas sp. 14og.

According to a further aspect of the present invention there is provided a method of treating or protecting a plant and/or plant derived materials from pest infestation wherein the method comprises applying to the plant or its environment an effective amount of a sonicated cell filtrate of Pseudomonas sp. 14og.

According to a further aspect of the present invention there is provided a method of treating or protecting a plant and/or plant derived from pest infestation wherein the method comprises applying to the plant or its environment a composition comprising an effective amount of Pseudomonas sp. 14og.

According to a further aspect of the present invention there is provided a method substantially as described above wherein the effective amount of Pseudomonas sp. 14og is obtained from a culture of Pseudomonas sp. 14og.

According to a further aspect of the present invention there is provided a method substantially as described above wherein the effective amount of Pseudomonas sp. 14og is obtained from a supernatant from a whole broth culture Pseudomonas sp. 14og.

According to a further aspect of the present invention there is provided a method of treating or protecting a plant and/or plant derived materials from pest infestation wherein the method comprises applying to the plant or its environment a composition comprising an effective amount of a cellular extract of Pseudomonas sp. 14og.

According to a further aspect of the present invention there is provided a method of treating or protecting a plant and/or plant derived materials from pest infestation wherein the method comprises applying to the plant or its environment a composition comprising an effective amount of a sonicated cell filtrate of Pseudomonas sp. 14og.

According to a further aspect of the present invention there is provided a method of controlling and/or preventing a pest infestation characterized by the step of applying a composition comprising an effective amount of Pseudomonas sp. 14og to a surface.

According to a further aspect of the present invention there is provided a method as claimed in claim 1, wherein the effective amount of Pseudomonas sp. 14og is obtained from a culture of Pseudomonas sp. 14og.

According to a further aspect of the present invention there is provided a method, as claimed in claim 1, wherein the effective amount of Pseudomonas sp. 14og is obtained from a supernatant from a whole broth culture of Pseudomonas sp. 14og.

According to a further aspect of the present invention there is provided a method of controlling and/or preventing a pest infestation characterized by the step of applying a composition comprising an effective amount of a cellular extract of Pseudomonas sp. 14og to a surface.

According to a further aspect of the present invention there is provided a method of controlling and/or preventing a pest infestation characterized by the step of applying a composition comprising an effective amount of a sonicated cell filtrate of Pseudomonas sp. 14og to a surface.

According to a further aspect of the present invention there is provided a method of controlling and/or preventing a pest infestation characterized by the step of applying a composition as substantially described above, to a surface.

According to a further aspect of the present invention there is provided the use of an isolated Pseudomonas sp. 14og bacterium deposited into the NRRL Patent Culture Collection on 3 Dec. 2020 and designated NRRL accession no. B-67999, or culture thereof, for use in the biopesticide activity against pest populations of insect species belonging to the order of Lepidoptera, Coleoptera, Diptera, Hemiptera, and/or the larvae thereof.

It should be appreciated by those skilled in the art that Pseudomonas sp. 14og has a biopesticide activity of broad application.

In one example, the biopesticide activity may be for the application against pest populations of insect species belonging to the order of Lepidoptera, Coleoptera, Diptera, Hemiptera, and/or the larvae thereof. Alternatively, the biopesticide activity may be for the application against fungal, nematode and mite pest populations. As used herein the term “isolated” means removed from the natural environment in which the bacteria naturally occurs and is separated from some or all of the co-existing materials in the natural system from which the bacteria has been obtained. As used herein the term “biopesticide” refers to a biologically derived substance having the ability to kill, or retard the growth of, insects and/or the larvae thereof. In particular, a biopesticide of the present invention should be capable of retarding growth, or killing, one or more of the insect species belonging to the order of Lepidopteran, Coleopteran, Dipteran, Hemipteran, and/or the larvae thereof. Alternatively, a biopesticide of the present invention should be capable of retarding growth, or killing, one or more fungal, nematode and mite pest populations. In one example, a biopesticide of the present invention should be capable of retarding the growth, or killing, at least one, but for example all or most of the species belonging to the orders of Lepidoptera, Coleoptera, Diptera, Hemiptera and/or the larvae thereof. Alternatively, a biopesticide of the present invention should be capable of retarding the growth, or killing, at least one, but for example all or most fungal, nematode and mite pest populations. As used herein the term “culture” refers to a population of bacteria together with the media in or on which the population was propagated (i.e. grown). As used herein the term “whole broth culture” refers to a liquid media and the population of bacteria therein.

In one embodiment the broth may be Luria-Bertani broth. However, it will be appreciated by a person skilled in the art that other suitable broths may be used. As used herein the term “cellular extract” refers to a substance or mixture of substances obtained from a bacterial cell. As used herein the term “sonicate” or grammatical variants thereof refers to subjecting a cell to ultrasonic vibrations in order to fragment the cell wall to release the contents of the cell. It should be appreciated that the “cellular extract” may be obtained in a variety of different ways, and may come in a variety of different forms without departing from the scope of the present invention.

In some embodiments the cellular extract may be a crude extract of the contents of the cell. In general the crude extract may be obtained via centrifugation of a whole broth culture re-suspended in a suitable buffer. Such an extract may have been derived from Sonication; French press; Mantin gaulin press, bead basher, bead mill, mincer, osmotic lysis and enzyme related lysis as known in the art.

In other embodiments the cellular extract may be a freeze dried or a spray dried extract. In general, the freeze or spray dried extract may be obtained via any cellular extract which has also been subjected to a freeze-or spray drying process or alternate processes as known in the art.

In one embodiment the cellular extract may be derived from the aforementioned methods via sonication; French press; Mantin gaulin press, bead basher, bead mill mincer osmotic lysis or enzyme related lysis. In general the inventors have found that a supernatant having a biopesticide activity will be obtained when the organism is grown in Luria-Bertani broth at 25° C. The term ‘plant’ refers to the plant in its entirety or a part thereof including selected portions of the plant during the plant life cycle such as the plant seeds, shoots, leaves, bark, pods, roots, flowers, stems and the like, including crop food and plant derived materials or parts thereof. The term ‘plant derived materials’ refers to products that may be produced from a plant or part thereof. It will be appreciated that a person skilled in the art will know of various examples of plant derived products, such as hay, silage or other types of feed or products. Compositions of the present invention may be formulated in a variety of different ways without departing from the scope of the present invention. In general the formulation chosen will be dependent on the end application. For example, possible formulations include, but should not be limited to:
Vectors such as the Trojan vector;
Matrixes;
Soluble powders;
Granules;
Micro encapsulation in a suitable medicine;
Aqueous suspensions;
Non-aqueous suspensions;
Emulsions;
Pastes;
Emulsifiable concentrations; or
Baits.

The present invention may include formulations suitable for:
direct application to insect affected areas e.g. drench, spray form;
suspended in a bait matrix;
slow release prills for subterranean applications; or
hydrophobic matrixes facilitating buoyancy for aquatic surface filter feeders.

It will be appreciated that other suitable formulations and/or methods of preparing the formulations and/or compositions will be known to those skilled in the art. The term bait as used herein refers to any foodstuff or other attractant to an insect or larvae thereof which includes an effective amount of:
a) Pseudomonas sp. 14og; or
b) a mutant or variant strain of Pseudomonas sp. 14og; or
c) a derivative of a) or b).

The term ‘effective amount’ as used herein refers to a suitable quantity for a biopesticide activity to be exhibited. The Pseudomonas sp. 14og of the present invention produces a biopesticide which can be applied directly to surfaces where insects may contact such as artificial/cultural surfaces (e.g. milled wood, concrete, and urban dwellings); as well as in agricultural systems such as plant surfaces seed coats or matter of plant origin. The present invention has application in both terrestrial and aquatic environments and may be applied in or on both soil and phylloplane or rhizosphere systems.

An embodiment of the present invention may have a number of advantages over the prior art which can include:
providing a new biopesticide;
providing a new method for controlling insects; and
providing a new biopesticide which has a range of different forms.

### BEST MODES FOR CARRYING OUT THE INVENTION

### Example 1 Physiological and Metabolic Characterization Gram Negative Rod

### Psychrotolerant Strain

Growth in LB media with subsequent plating, shows convex circular colony forms with high level of fluorescence are apparent. If the colonies are allowed to grow to over 3-4 days all colonies have exhibit a watery and ropey consistency indicative that it expresses a high level of exopolysaccharides and/or biofilm. The bacteria forms a viscous gel-like substance in LB culture grown at 28° C. after 48 hours. The bacteria produces creamy substance in minimal media culture grown at 28° C. after 48 hours. The bacteria shows reduced growth at 4° C. and 37° C. The bacteria shows optimal growth at 26° to 30° C. The bacteria may form swarming colonies between 19° C. and 33° C. in reduced agar media.

| Test | Result |
|---|---|
| Gram stain | Negative |
| Oxidase | Positive |
| Catalase | Positive |
| Motility | Positive |

The strains were tested using the commercial bacterial identification system, Biolog Gen III Microbial ID system. Results are shown in Table 2 below.

TABLE 2
Testing results from Biolog Gen III Microbial
ID system bacterial identification system

| Code | Substrate | Result at 24 hours |
|---|---|---|
| A1 | Negative Control | − |
| B1 | D-Raffinose | − |
| C1 | α-D-Glucose | + |
| D1 | D-Sorbitol | − |
| E1 | Gelatin | − |
| F1 | Pectin | − |
| G1 | p-Hydroxy-Phenylacetic acid | + |
| H1 | Tween 40 | − |
| A2 | Dextrin | + |
| B2 | α-D-Lactose | − |
| C2 | D-Mannose | + |
| D2 | D-Mannitol | + (weak) |
| E2 | Glycyl-L-Proline | − |
| F2 | D-Galacturonic Acid | − |
| G2 | Methyl Pyruvate | − |
| H2 | γ-Amino-Butyric Acid | + (weak) |
| A3 | D-Maltose | − |
| B3 | D-Melibiose | − |
| C3 | D-Fructose | + |
| D3 | D-Arabitol | − |
| E3 | L-Alanine | + |
| F3 | L-Galactonic Acid Lactose | − |
| G3 | D-Lactic Acid Methyl Ester | − |
| H3 | α-Hydroxy-Butyric Acid | − |
| A4 | D-Trehalose | + |
| B4 | β-Methyl-D-Glucoside | − |
| C4 | D-Galactose | + |
| D4 | myo-Inositol | − |
| E4 | L-Arginine | + |
| F4 | D-Gluconic Acid | + |
| G4 | L-Lactic Acid | + |
| H4 | α-Hydroxy-D,L-Butyric Acid | − |
| A5 | D-Cellobiose | − |
| B5 | D-Salicin | − |
| C5 | 3-Methyl Glucose | − |
| D5 | Glycerol | − |
| E5 | L-Aspartic Acid | + |
| F5 | D-Glucoronic Acid | − |
| G5 | Citric Acid | + |
| H5 | α-Keto-Butyric Acid | − |
| A6 | Gentiobiose | − |
| B6 | N-Acetyl-D-Glucosamine | − |
| C6 | D-Fucose | + (weak) |
| D6 | D-Glucose-6-PO4 | − |
| E6 | L-Glutamic Acid | + |
| F6 | Glucoronamide | + |
| G6 | α-Keto-Glutaric Acid | + (weak) |
| H6 | Acetoacetic Acid | − |
| A7 | Sucrose | − |
| B7 | N-Acetyl-β-D-Mannosamine | − |
| C7 | L-Fucose | + |
| D7 | D-Fructose-6-PO4 | + (weak) |
| E7 | L-Histidine | + |
| F7 | Mucic Acid | + |
| G7 | D-Malic Acid | − |
| H7 | Propionic Acid | − |
| A8 | D-Turanose | − |
| B8 | N-Acetyl-D-Galactosamine | − |
| C8 | L-Rhamnose | − |
| D8 | D-Aspartic Acid | − |
| E8 | L-Pyroglutamic Acid | + (weak) |
| F8 | Quinic Acid | + |
| G8 | L-Malic Acid | + |
| H8 | Acetic Acid | + |
| A9 | Stachyose | − |
| B9 | N-Acetyl Neuraminic Acid | − |
| C9 | Inosine | − |
| D9 | D-Serine | − |
| E9 | L-Serine | + |
| F9 | D-Saccharic Acid | + (weak) |
| G9 | Bromo-Succinic Acid | − |
| H9 | Formic Acid | − |
| A10 | Positive Control | + |
| B10 | 1% NaCl | + |
| C10 | 1% Na Lactate | + |
| D10 | Troleandomycin | + |
| E10 | Lincomycin | + |
| F10 | Vancomycin | + |
| G10 | Nalidixic Acid | + (weak) |
| H10 | Aztreonam | + |
| A11 | pH 6 | + |
| B11 | 4% NaCl | − |
| C11 | Fusidic Acid | + |
| D11 | Rifamycin SV | + |
| E11 | Guanidine HCl | + |
| F11 | Tetrazolium Violet | + |
| G11 | Lithium Cloride | − |
| H11 | Na Butyrate | − |
| A12 | pH 5 | + (weak) |
| B12 | 8% NaCl | − |
| C12 | D-Serine | + (weak) |
| D12 | Minocylcine | − |
| E12 | Niaproof 4 | + |
| F12 | Tetrazolium Blue | + |
| G12 | Potassium Tellurate | − |
| H12 | Na Bromate | − |

Score at 24 hours denotes the genus is Pseudomonas, but the species is inconclusive.

Mortality with wild-type Pseudomonas 14og was 71.4% (Table 1).
We further prepared different filtered fractions of Pseudomonas 14og and found that at least one active toxin passed through a 1 kilodalton membrane filter.
We further prepared a precipitate of Pseudomonas 14og and found that at least one active toxin was precipitated by the addition of ammonium sulfate.

TABLE 3

| | | | P value |
|---|---|---|---|
| | % Treated | | (two |
| Treatment^{a} | Mortality(n)^{b} | | tailed)^{c} |
| Control ^{d} | 0% | (0/21) | P < 0.05 |
| Whole broth culture of Pseudomonas 14og | 71.4% | (15/21) | P < 0.05 |
| Concentrated filtrate of Pseudomonas | 71.4% | (15/21) | P < 0.05 |
| 14og | | | |
| Concentrated precipitate of Pseudomonas | 85.7% | (18/21) | P < 0.05 |
| 14og | | | |

Third instar larvae of healthy Japanese beetles were submerged in a solution of Pseudomonas 14og whole broth culture, or the filtered and concentrated fraction or a solution containing the concentrated precipitate of Pseudomonas 14og whole broth culture
^{b}Mortality is the percentage of larvae death. n is the sample size of the treated groups. The data were collected on day 20.
^{c}The two tail student t-test was used to elucidate statistical significance. Each treatment was repeated three times.
^{d} Similar to b, n is the sample size of the negative control saline-treated group.

TABLE 4

| | | | P value |
|---|---|---|---|
| | % Treated | | (two |
| Treatment^{a} | Mortality(n)^{b} | | tailed)^{c} |
| Control ^{d} | 0% | (0/21) | P < 0.05 |
| Whole broth culture of Pseudomonas 14og | 71.4% | (15/21) | P < 0.05 |
| Concentrated filtrate of Pseudomonas | 71.4% | (15/21) | P < 0.05 |
| 14og | | | |
| Concentrated precipitate of Pseudomonas | 100% | (21/21) | P < 0.05 |
| 14og | | | |

Third instar larvae of healthy Northern Masked Chafer beetles were submerged in a solution of Pseudomonas 14og whole broth culture, or the filtered and concentrated fraction or a solution containing the concentrated precipitate of Pseudomonas 14og whole broth culture
^{b}Mortality is the percentage of larvae death. n is the sample size of the treated groups. The data were collected on day 20.
^{c}The two tail student t-test was used to elucidate statistical significance. Each treatment was repeated three times.
^{d} Similar to b, n is the sample size of the negative control saline-treated group.

The results presented in TABLE 3 and TABLE 4 expressed as percent of grubs that have died, indicate that the extracts have a significantly toxic effect on Popilliae japonica and Cyclocephala borealis. The extract of Pseudomonas 14og, induces a mortality of 71.4% and 100%, respectively.
We further prepared chromatographic fractions from a culture of Pseudomonas 14og and found that at least one active toxin was able to be dried and concentrated at ambient temperature and pressure.
We further purified one particular fraction which was found to be efficacious against Japanese beetle larvae.

TABLE 5

| | % Treated | |
|---|---|---|
| Treatment^{a} | Mortality(n)^{b} | |
| Control^{c} | 0% | (0/7) |
| 4.9 mg/ml Pseudomonas 14og chromatographic extract | 100% | (7/7) |

Third instar larvae of healthy Japanese beetles were submerged in a solution containing purified Pseudomonas 14og chromatographic extract.
^{b}Mortality is the percentage of larvae death. n is the sample size of the treated groups. The data were collected on day 15.
^{c}Similar to b, n is the sample size of the negative control saline-treated group.

The results presented in TABLE 5 expressed as percent of grubs that have died, indicate that the extracted fraction has a significantly toxic effect on Popilliae japonica. The extract of Pseudomonas 14og, induced a mortality of 100%.
The active chromatographic extract was further analyzed with mass spectroscopy and determined to contain a molecule having a monoisotopic mass of 1148.68 Da. Further analysis of this molecule revealed a possible peptide sequence of Gly Val Leu/Ile Ser Leu/Ile which may correspond with a novel viscosinamide, pseudophomin, massetolide, or part of a similar cyclic lipopeptide.
At least one other chromatographic fraction from a culture of Pseudomonas 14og was found that contained at least one other active toxin which was able to be dried and concentrated at ambient temperature and pressure.
The second active chromatographic extract was further analyzed with mass spectroscopy and determined to contain a molecule having a monoisotopic mass of 1184.68 Da.
At least one other chromatographic fraction from a culture of Pseudomonas 14og was found that contained at least one other active toxin which was able to be dried and concentrated at ambient temperature and pressure.
The third active chromatographic extract has not yet been further analyzed.

### Example 2: Genetic Identification

The inventors have shown the Pseudomonas sp. 14og is a member of the Pseudomonas fluorescens complex, and that phenotypically, Pseudomonas sp. 14og is most similar to P. marginalis or P. putida. However, genetic characterization places it in a separate, unique biotype or species. There exists a small cluster of species which may in fact comprise a single species which is closely related to Pseudomonas yamanorum.

Although, it is beyond the scope of this application to correct taxonomic assignments or redefine the current Pseudomonas fluorescens complex or subgroups which comprise wide genetic variation and many ambiguously defined entries, for the purpose of this application the following isolates, species or strains are also claimed herein as being members of the same taxonomic species as Pseudomonas sp. 14og:
Pseudomonas fluorescens BBc6R8
Pseudomonas sp. SXM-1
Pseudomonas sp. NC02
Pseudomonas sp. Colony2

The inventors also seek to include any biotypes, and/or strains, and/or progeny and/or isolates derived from the species, Pseudomonas sp. 14og.

The inventors also seek to include any biotypes, and/or strains, and/or progeny and/or isolates derived from the species closely related to Pseudomonas sp. 14og.

The inventors also seek to include any altered variants and/or progeny and/or isolates derived from Pseudomonas sp. 14og and/or species closely related to Pseudomonas sp. 14og.

Altered variants of Pseudomonas sp. 14og may be created by mutating the cells or genes via numerous methods known in the state of the art. Altered variants of Pseudomonas sp. 14og may differ by substituted nucleotides which do not affect the resultant amino acid sequence which may be produced by a particular gene.

More particularly, the inventors also seek to include any organism having 98% or greater genetic relatedness to Pseudomonas sp. 14og.

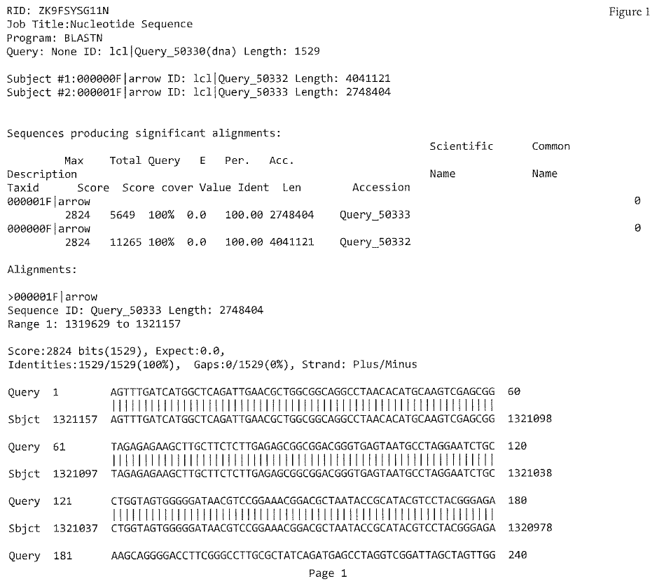

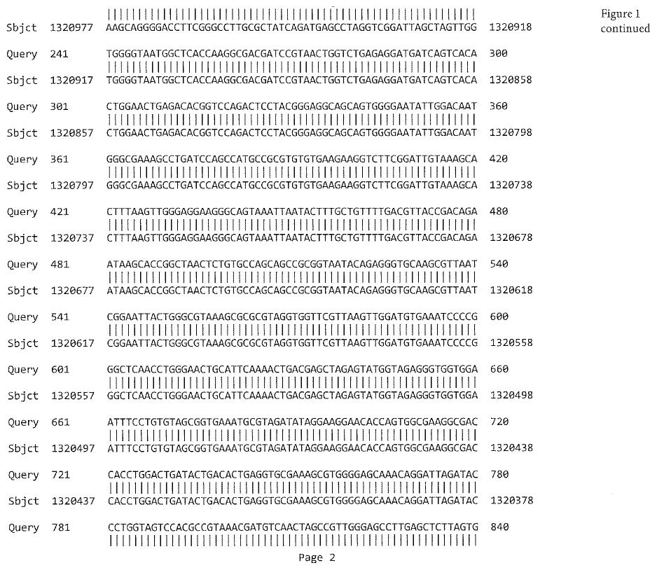

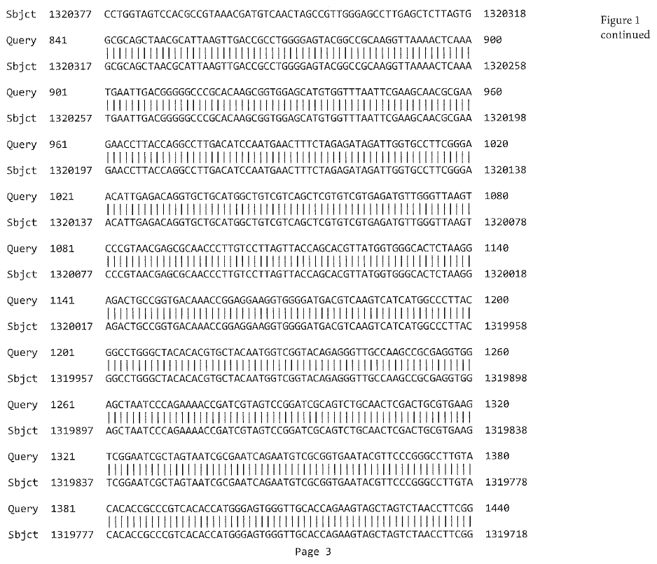

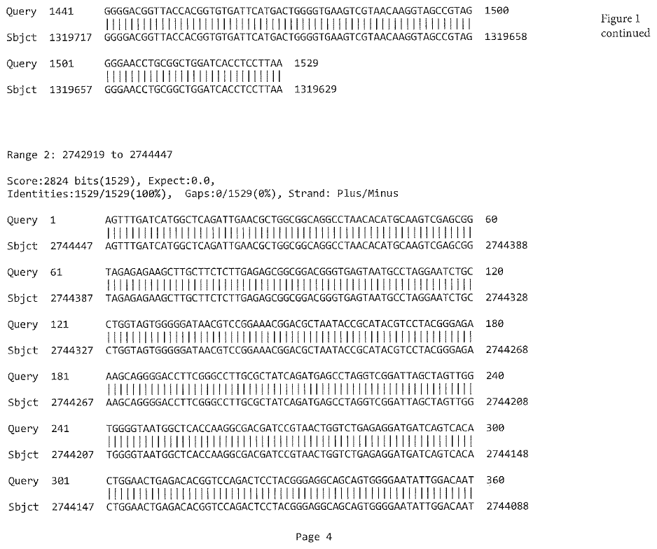

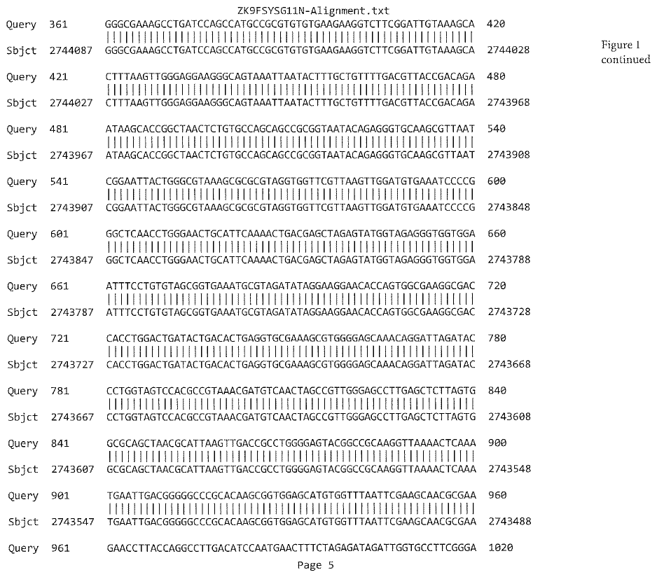

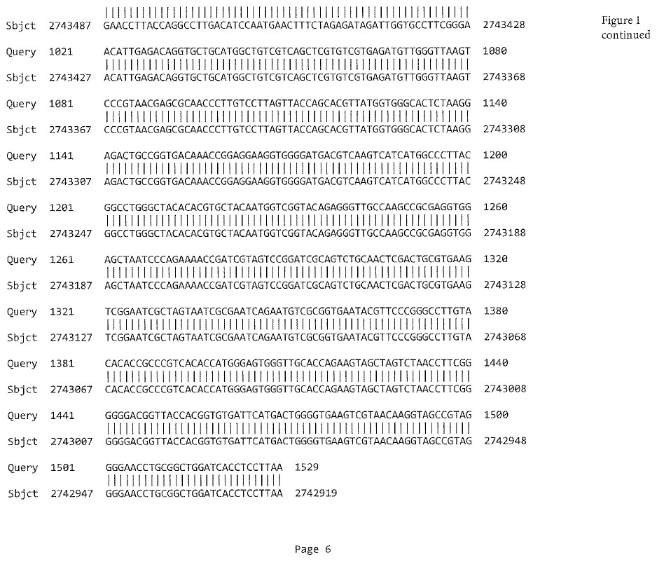

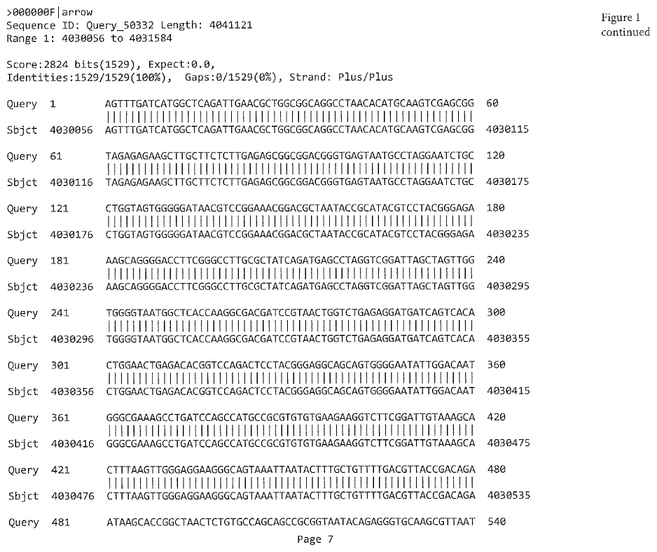

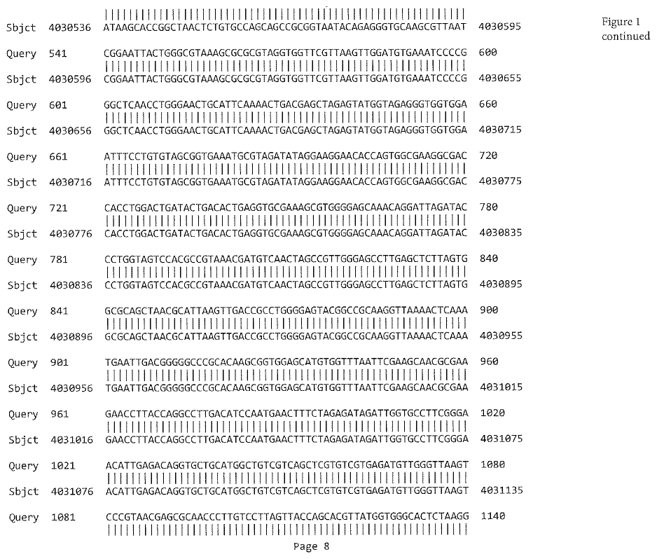

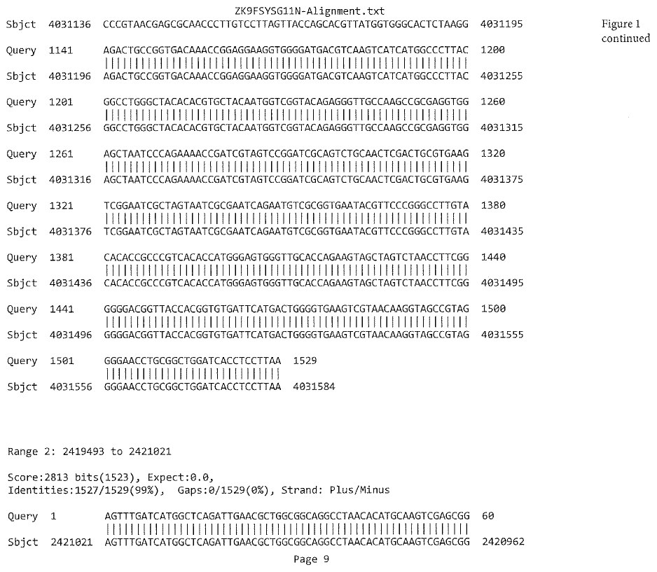

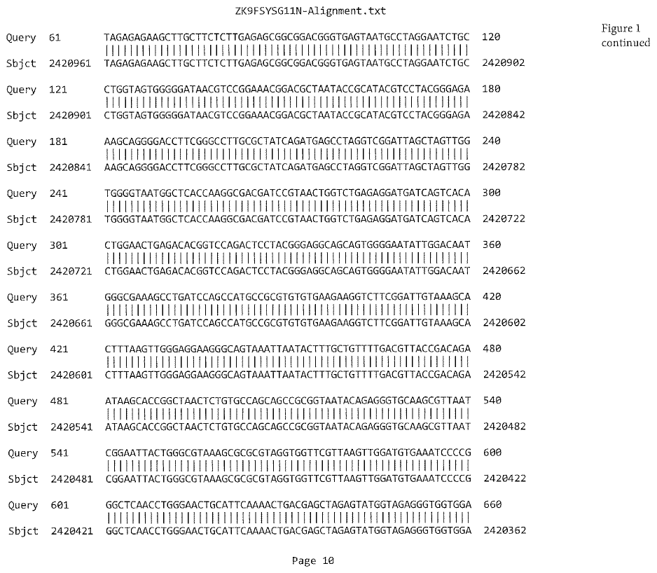

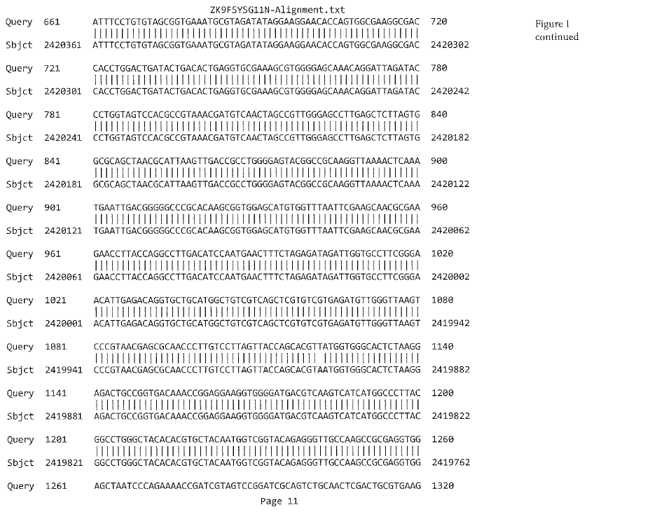

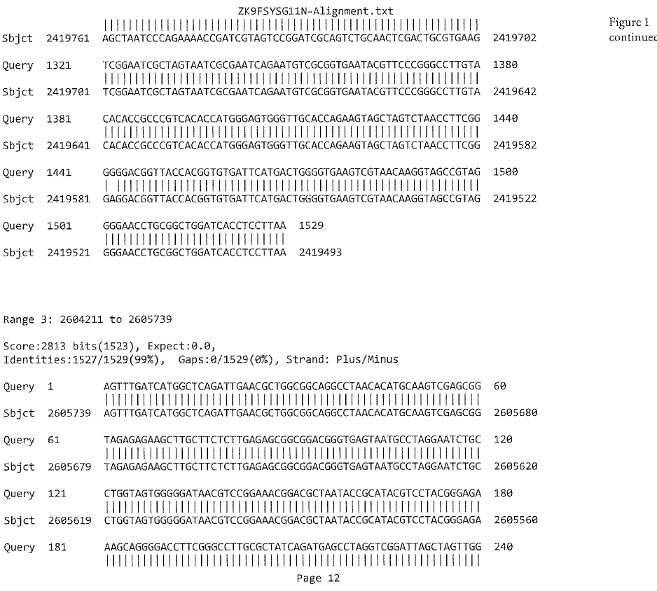

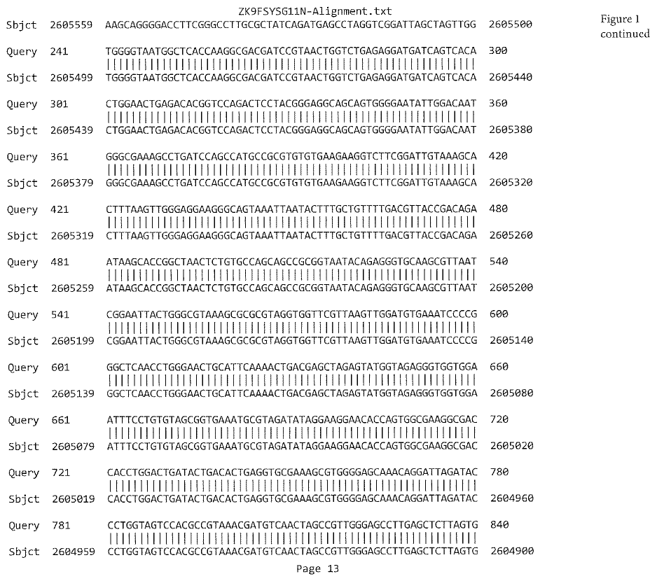

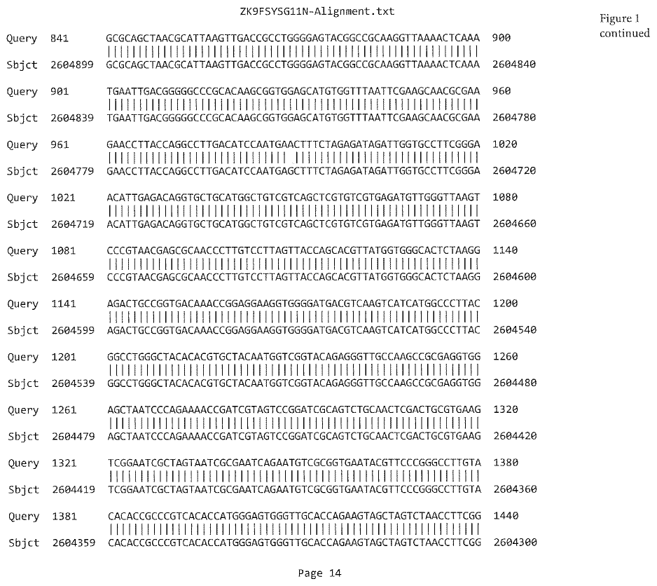

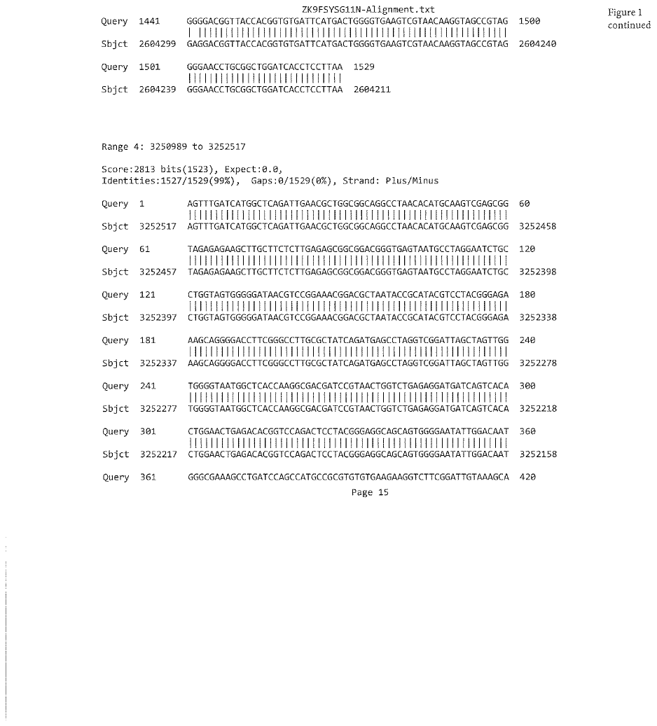

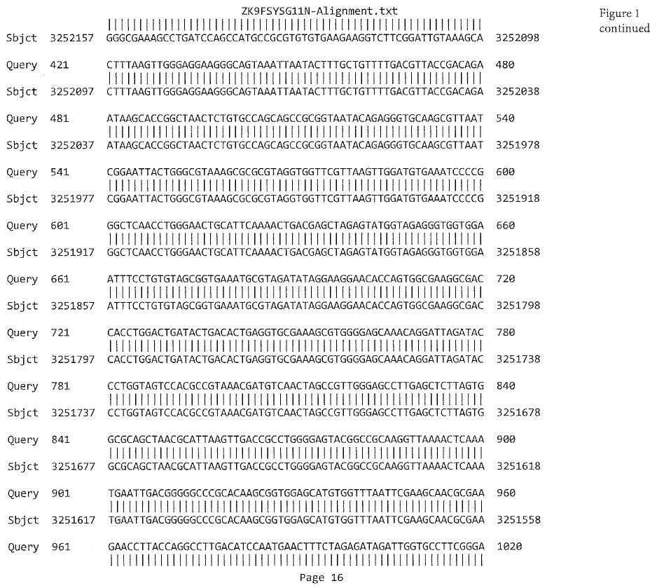

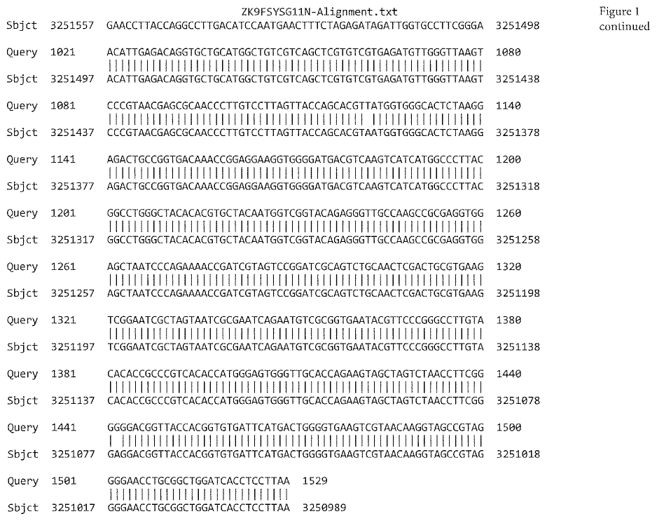

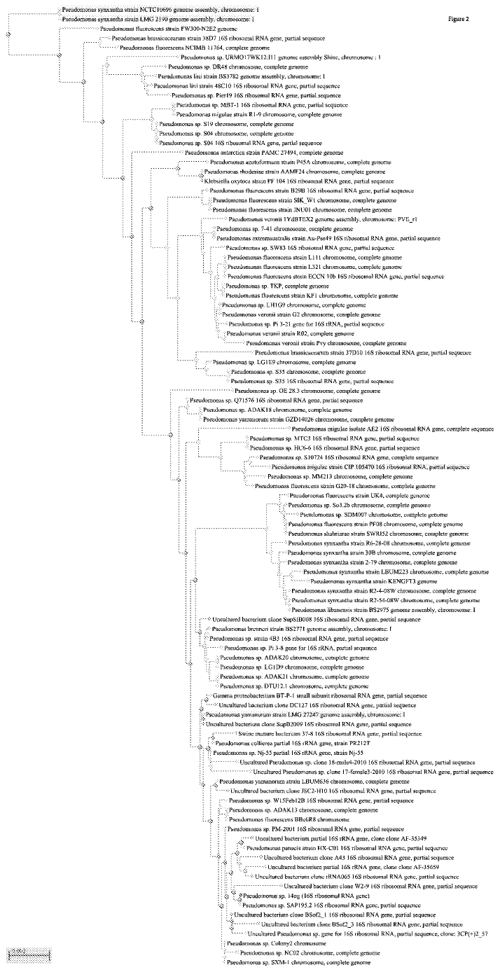

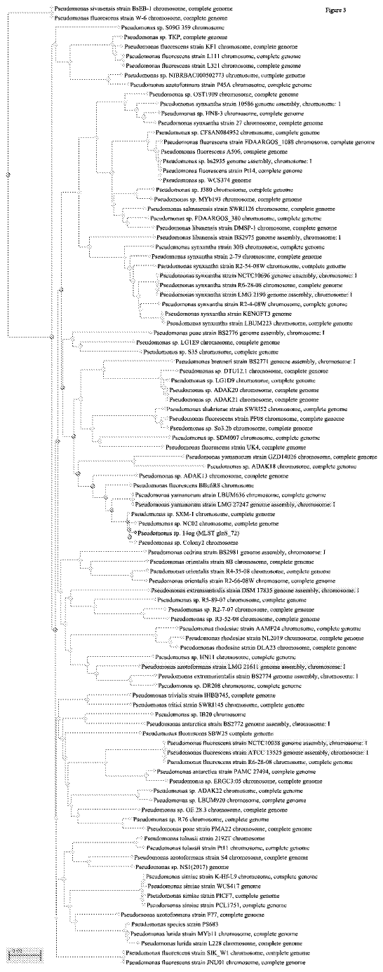

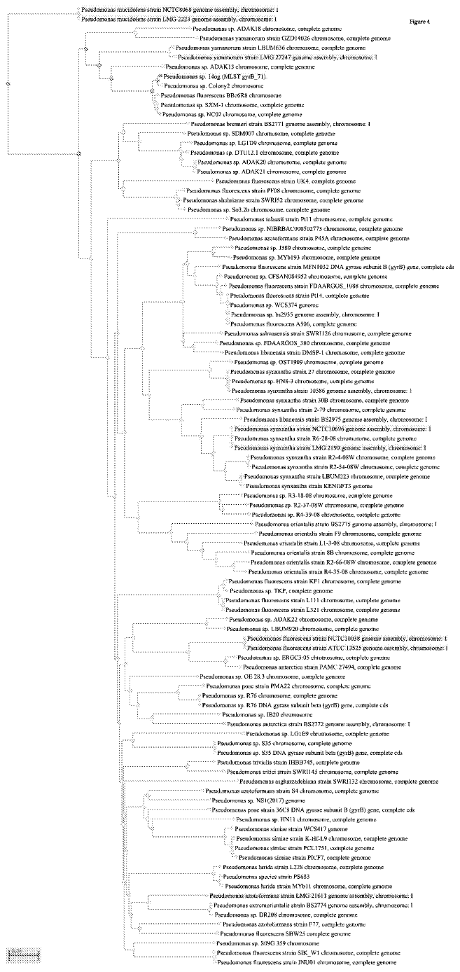

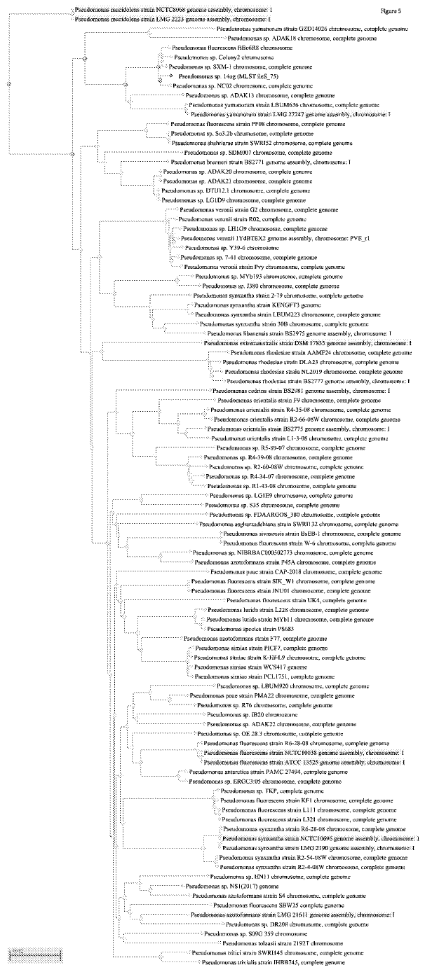

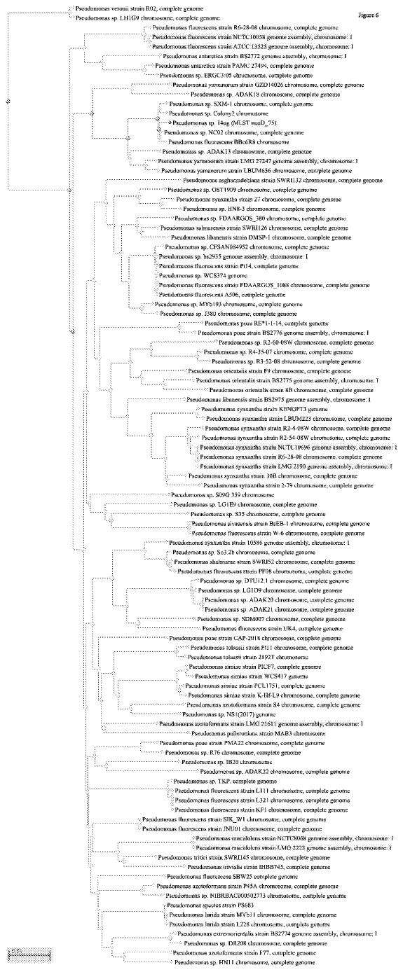

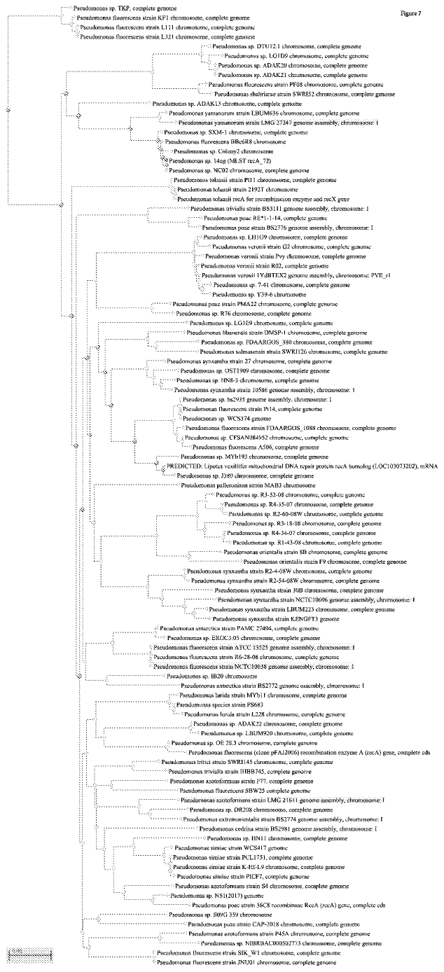

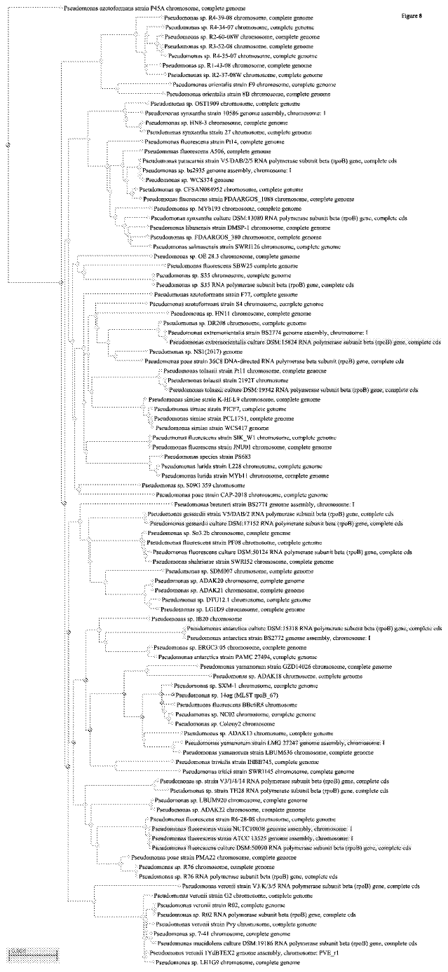

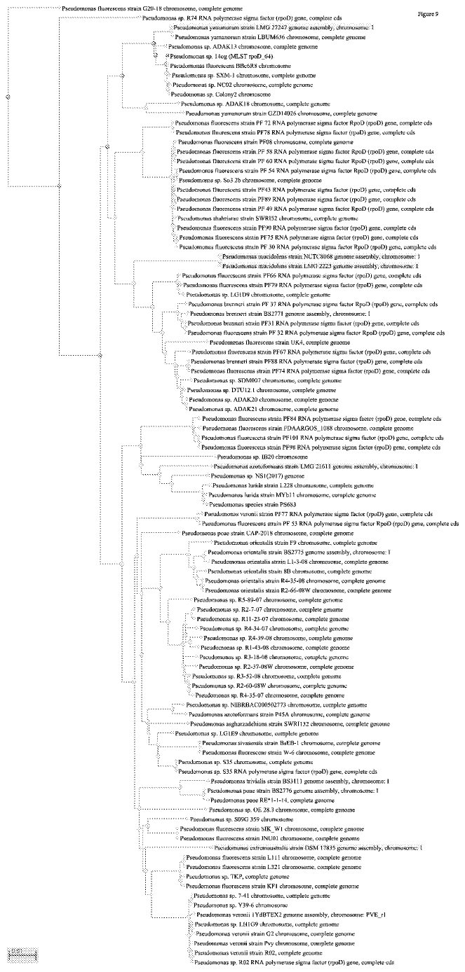

More particularly, the inventors also seek to include any organism having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or at least 99.8% similar genetic sequences as provided in the sequences listed in 1234WOPCT_SequenceListing.txt which represents the nucleotide sequences of glnS, and/or gyrB, and/or ileS, and/or nuoD, and/or recA, and/or rpoB, and/or rpoD, and/or similar genetic sequences as provided in the sequences listed in FIG. 1, which represent the nucleotide sequences of the 16s rRNA gene found in Pseudomonas sp. 14og.

The percentage identity may be at least 90%. In one example the percentage identity will be at least 95%. For example, 98% the same DNA of the invention may comprise an amino acid sequence having a 100% identity with glnS, and/or gyrB, and/or ileS, and/or nuoD, and/or recA, and/or rpoB, and/or the rpoD gene found in Pseudomonas sp. 14og.

Progeny bacteria and proteins and/or peptides and/or metabolites produced using Pseudomonas sp. 14og may be identified, for example, by having a molecular marker profile of at least 25%, 30%, 35%, 40%, 45%, 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% genetic contribution from Pseudomonas sp. 14og, as measured by either percent identity or percent similarity. Progeny may be further characterized as being within a pedigree distance of Pseudomonas sp. 14og, such as within 1, 2, 3, 4, or 5 or less conjugations with Pseudomonas sp. 14og, or a bacteria that has Pseudomonas sp. 14og as a progenitor. Unique molecular profiles may be identified with other molecular tools, such as restriction fragment length polymorphisms, also known as RFLPs, or single nucleotide polymorphisms, also known as SNPs.

1. Pseudomonas sp. 14og a bacteria of NRRL accession no. B-67999.

2. A plant pest control agent comprising a bacterium according to claim 1, wherein said plant pest control agent kills plant pests by toxins secreted by said bacterium.

3. The plant pest control agent according to claim 2, wherein the plant pest is an insect of Coleoptera.

4. A plant pest control agent according to claim 3, wherein said beetle insects are selected from the genus consisting of Popilliae, Anomala, Cyclocephala, Melolontha, Amphimallon or Phyllophaga.

5. The plant pest control agent of claim 2, wherein said plant pest is selected from Lepidopteran, Coleopteran, Dipteran, Hemipteran, Tetranychidae, fungal, nematode and/or mite pest populations.

6. A whole culture comprised of claim 1, wherein the culture is used as a crop protectant and/or pesticide.

7. A whole culture comprised of claim 1, wherein the culture is used as an adjuvant in conjunction with another crop protectant and/or pesticide.

8. A whole culture comprised of claim 1, wherein the culture is used as a crop growth adjuvant and/or in conjunction with another crop growth adjuvant.

9. An extract and/or filtrate of the bacterium according to claim 1, wherein said plant pest control agent kills plant pests by toxins extracted from said bacterium.

10. An extract and/or filtrate of a secretion of the bacterium according to claim 2, wherein said plant pest control agent kills plant pests by toxins extracted from said secretion of the bacterium.

11. An extract and/or filtrate of a whole culture of the bacterium according to claim 6, wherein said extract and/or filtrate contains toxins extracted from said culture of the bacterium.

12. An extract and/or filtrate of a whole culture of the bacterium according to claim 7, wherein said extract and/or filtrate contains toxins extracted from said culture of the bacterium.

13. An extract and/or filtrate of a whole culture of the bacterium according to claim 8, wherein said extract and/or filtrate contains toxins extracted from said culture of the bacterium.

14. An extract and/or filtrate comprised of claim 9, wherein the extract and/or filtrate is used as a crop protectant and/or pesticide.

15. An extract and/or filtrate comprised of claim 10, wherein the extract and/or filtrate is used as a crop protectant and/or pesticide.

16. An extract and/or filtrate comprised of claim 11, wherein the extract and/or filtrate is used as a crop protectant and/or pesticide.

17. An extract and/or filtrate comprised of claim 9, wherein the extract and/or filtrate is used as an adjuvant in conjunction with another crop protectant and/or pesticide.

18. An extract and/or filtrate comprised of claim 10, wherein the extract and/or filtrate is used as an adjuvant in conjunction with another crop protectant and/or pesticide.

19. An extract and/or filtrate comprised of claim 11, wherein the extract and/or filtrate is used as an adjuvant in conjunction with another crop protectant and/or pesticide.

20. An extract and/or filtrate comprised of claim 9, wherein the extract and/or filtrate is used as a crop growth adjuvant and/or in conjunction with another crop growth adjuvant.

21. An extract and/or filtrate comprised of claim 10, wherein the extract and/or filtrate is used as a crop growth adjuvant and/or in conjunction with another crop growth adjuvant.

22. An extract and/or filtrate comprised of claim 11, wherein the extract and/or filtrate is used as a crop growth adjuvant and/or in conjunction with another crop growth adjuvant.

23. A method for preparing a non-ribosomal peptide compound, wherein the non-ribosomal peptide compound is obtained by liquid fermentation culture, and/or solid phase extraction technology and/or high performance liquid chromatography separation of Pseudomonas sp. 14og according to claim 1.

24. Use of a composition comprising a lipopeptide from Pseudomonas sp. 14og according to claim 1, and/or viscosinamide-family of lipopeptides for pest control, wherein the effective content of the lipopeptide in the composition is 0.01%-99%, and the pest is selected from Lepidopteran, Coleopteran, Dipteran, Hemipteran, Tetranychidae, fungal, nematode and/or mite pest populations.


SEQUENCE LISTING (7 sequences)

SEQ ID NO: 1 - DNA, 1701 bp, Pseudomonas sp.
  source 1..1701: mol_type = genomic DNA
atgagcaagcccactgtcgaccctacctcgaattccaaggccggacctgccgtcccggtcaatttcctgcgcccgatcatccaggcggacctggattcgggcaagcacacgcagatcgtcacccgcttcccgcctgagcccaacggttacctgcacatcggccacgccaagtcgatctgtgtgaacttcggcctggcccaggagttcggtggcgtcacgcacctgcgtttcgacgacaccaacccggccaaggaagaccaggaatacatcgacgccatcgaaagcgacatcaagtggctgggcttcgaatggtccggtgaagtgcgctatgcctccaagtatttcgaccagttgttcgactgggccgtcgagttgatcaaggccggcaaggcctacgtagacgacctgaccccggagcaagccaaggaataccgtggcaccctgaccgagccgggcaagaacagcccgttccgcgaccgttcggttgaagagaacctggactggttcaaccgcatgcgcgccggcgagttcccggacggcgcccgcgtactgcgtgccaagatcgacatggcctcgccgaacatgaacctgcgcgacccgatcatgtaccgcatccgccacgcccatcaccaccagaccggtgacaagtggtgcatctacccgaactacgacttcacccacggtcagtcggacgccatcgaaggcatcacccactccatctgcaccctggagttcgaaagccaccgtccgctgtacgaatggttcctcgacagcctgccggtaccggcgcacccgcgtcagtacgagttcagccgcctgaacctgaactacaccatcaccagcaagcgcaagctcaagcaactggtcgatgaaaagcacgtgtttggctgggacgacccgcgtatgtcgacgctgtcgggcttccgccgccgtggatacaccccggcatcgatccgcaatttctgcgagatggtcggcaccaaccgctccgacggcgtggtcgactacggcatgctcgaattcagcatccgtcaggacctcgacgcgaacgccccgcgcgccatgtgcgtgctgcgtccgttgaaggtggtgatcaccaactacccggaaggccaggtcgaaaacctcgaactgccacgtcatccgcagaaagaagaactcggcgtgcgcaagctgccgttcgcccgtgaaatctacatcgaccgcgatgacttcatggaagagccgccaaaaggctacaagcgcctggagccgaacggcgaagtacgcctgcgcggcagctacgtgatccgtgccgacgaagcgatcaaggacgccgatggcaacatcgtcgaactgcgttgctcctacgacccggacaccctgggcaagaacccggaaggccgcaaggtcaaaggtgtggtgcactgggtgccggcggcggccagcgtcgagtgcgaagtgcgcctgtacgatcgcctgttccgctcgccgaatcccgagaaggccgaagacagtgccagtttcctggacaacatcaaccctgactcgctgcaagttctcacgggttgtcgtgccgagccatcgcttggcgacgcacagccggaagaccgtttccagttcgagcgcgaaggttacttctgcgcggatatcaaggactcgaaacccggtgctccggtattcaaccgtaccgtgaccttgcgtgattcgtggggccagtga

SEQ ID NO: 2 - DNA, 2424 bp, Pseudomonas sp.
  source 1..2424: mol_type = genomic DNA
atgagcgaagaaaatacgaatacgtacgactcgaccagcattaaagtgcttaaaggtttggatgccgtacgcaaacgtcccggtatgtacatcggtgataccgatgatggcagcggtctgcaccacatggtgttcgaggtggtcgataactcgatcgacgaagctttggccggtcactgcgacgacatcagcatcatcatccacccggacgaatccatcaccgtacgcgacaacggtcgcggcattccggtagatgtgcataaagaagaaggcgtttcggcggcagaggtcatcatgaccgtgctccacgccggcggtaagttcgacgataactcctataaagtatccggcggtttgcacggtgtaggtgtttcggtagtaaacgccctttctgaagagctgatcctgactgtacgtcgtagcggcaagatctgggaacagacttacgttcatggtgttccacaggaaccgatgaaaatcgtcggtgacagcgaaagcactggtacgcagattcacttcaagccttcggctgaaaccttcaagaacatccatttcagctgggacattctggccaagcggattcgtgaactgtctttcctgaactccggtgtgggtatcgttctcaaggacgaacgcagcggcaaggaagagctgttcaagtacgaaggcggcctgcgtgcgttcgttgaatacctgaacaccaacaagaccgcggtcaatcaggtgttccacttcaacatccagcgtgaagacggcatcggcgtagaaatcgccctgcagtggaacgacagcttcaacgagaacctgttgtgcttcaccaacaacattcctcagcgcgatggcggtactcacctggtgggtttccgttctgcactgacgcgtaacctgaacacctacatcgaagcggaaggcttggcgaagaaacacaaagtcgccaccaccggtgacgatgcccgcgaaggcctgaccgccattatctcggtcaaggttccggatccgaagttcagctcccagaccaaagacaagctggtgtcctccgaagtgaagacagcggtcgaacaggaaatgggcaagtacttctccgacttcctgctggaaaacccgaacgaagccaagctggttgtcggcaagatgatcgacgcagcacgggctcgtgaagcggcgcgtaaagcccgtgagatgacgcgtcgtaaaggtgcgctggatatcgccggcctgccgggcaaactggctgactgccaggagaaagaccctgccctctccgaactgtacctggtggaaggtgactctgctggcggttccgccaagcagggtcgcaaccgtcgcacccaggccatcctgccgttgaagggtaagatcctcaacgtcgagaaggcgcgtttcgacaagatgatttcctcgcaggaagtcggcaccttgatcaccgccctgggctgtggcatcggccgcgatgagtacaacatcgacaagctgcgctaccacaacatcatcatcatgaccgatgctgacgttgacggttcgcacatccgcaccctgctgctgacgttcttcttccgtcagttgccggagttgatcgagcgtggctacatctacatcgcccagccgccgttgtacaaagtgaaaaaaggcaagcaagagcaatacatcaaagacgacgacgccatggaagagtacatgacgcagtcggccctggaagatgcgagcctgcacttgaacgacgaagccccgggcatctccggtgaagcgttggagcgcctggtaaacgacttccgcatggtgatgaagaccctcaagcgtttgtcgcgcctgtacccccaggagctgaccgagcacttcatctacctgccatccgtcagcctggaacagttgggtgatcatgcccacatgcaggcttggttggcccagtacgaagtccgcctgcgtaccgttgaaaaatccggcctggtctacaaagccagcctgcgtgaagaccgtgaacgtaacgtctggctgccagaggtcgaactgatctcccacggcctgtcgaactacgtcaccttcaaccgcgacttcttcggcagcaacgactacaagacggttgtaaccctcggcgcgcaactgagcaccctgctggacgacggtgcctacattcagcgtggcgagcgcaagaaagcggtcaaagagttcaaggaagccctggactggttgatggcggaaagcaccaagcgccataccattcagcgatacaaaggtctgggcgaaatgaacccggatcagctgtgggaaaccaccatggacccagcccagcgtcgcatgctgcgcgtgaccatcgaagacgccattggcgcggaccagatcttcaacaccctgatgggtgatgcggtcgagcctcgtcgtgacttcatcgagagcaatgccctggcggtttccaacctggacttctga

SEQ ID NO: 3 - DNA, 2784 bp, Pseudomonas sp.
  source 1..2784: mol_type = genomic DNA
atgaaggccggcctgccacagcgcgaaccgcagatcctgcagcgctgggacagtattggcctgtacggaaagttgcgcgagattggcaaggatcgtccgaaattcgtcctgcacgacggccctccttatgccaacggcacgattcacatcggtcatgcgctgaacaagattctcaaggacatgatcatccgctcgaaaaccctttcgggtttcgacgcgccttatgtcccgggttgggactgccacggcctgccgattgaacacaaggtcgaagtgacctacggcaagaacctgggcgcggataaaacccgcgaactgtgccgtgcctacgccaccgagcagatcgaagggcagaagtccgaattcatccgcctgggggtgttgggcgactgggccaacccgtacaagaccatgaacttcaagaacgaggctggtgaaatccgcgccttggccgaaatcgtcaagggcggtttcgtgttcaagggcctcaagcccgtgaactggtgcttcgattgcggctcggccctggctgaagccgaagtcgagtacgaagacaagaagtcctcgaccatcgacgtggccttcccgatcgccgacgaagccaaactggcgcaagccttcggtctggcgagcctggccaagccagcctctattgtgatctggaccaccaccccgtggaccatcccggccaaccaggcgctgaacgtgcacccggaattcacctacgccctggtggacgtcggtgacaaactgctggtgctggctgaagagctggtggaggcgtgcctggcccgctacaacctgcaaggctcggtgatcgcgaccactaccgggtccgcgctggaactgatcaacttccgtcacccgttctacgaccgcctgtcgccaatctacctggccgagtacgttgagctgggcgccggcaccggcgtggttcactgctcgcctgcctatggcgttgacgacttcgtgatctgcaagcagtacggcctggtcaacgacgacatcatcaatcccgtgcaaagcaacggcgtgtacgtgccgtccctggaattcttcggtggccagttcatcttcaaggccaacccggcgatcgtcgacaagctggcggaagtcggtgcgctgctgcacaccgaaaccatcacccacagctacatgcattgctggcgtcacaagaccccgctgatctaccgcgccactgcgcagtggtttatcggcatggacaaggagcctacccgcggcgaaaccctgcgcaagcgtgcgatcaaggcgatcgaagacaccaagttcgttccggcctggggccaggcgcgcctgcactcgatgatcgccaaccgtccggactggtgcatctcccgccagcgcaactggggcgtgccgattccgttcttcctgaacaaggaaagcggcgagttgcacccacgtaccgtcgagctgatggaagaggttgcccagcgcgttgaacaagagggcatcgaagcctggttcaagctggacgccgccgagttgctgggcgacgaagcgccgctgtacgacaagatcagcgacaccctcgacgtctggttcgactcgggtaccacccactggcacgtactgcgcggctcgcacccgatgggccacgagaccggcccgcgtgccgacctgtacctggaaggttcggaccaacaccgtggctggttccactcctcgttgctgaccggttgcgcgatcgacgatcacgccccgtaccgcgagctgctgacccacggtttcaccgtcgacgagaacggtcgcaagatgtccaagtccctgggcaacgtgatcgcgccgcaaaaggtcaacgacaccctgggcgccgacatcatgcgcctgtgggttgcctctaccgattactcgggcgagatggccgtttccgagcagatcctgcagcgcagcgccgatgcctaccgccggatccgtaataccgcacgcttcatgctgtcgaacctgaccggtttcaacccggccaccgacatcctgccggccgaggaaatgctcgccctggaccgttgggccgtggaccgtaccctgttgctgcagcgcgagctgcaagagcactacggcgaataccgcttctggaacgtctactcgaagatccacaacttctgcgtgcaggagctgggtggtttctacctcgacatcatcaaggaccgccagtacaccaccggcgccaacagcaaggcccgccgttcggcgcagaccgcgctgtaccatatctctgaagcgctggtgcgctggatcgccccgatcctggcattcaccgccgacgaactgtgggaatacctgccgggcgagcgtaacgagtccgtgatgctcaacacctggtacgaaggcctgaccgaattgccggccgacttcgagctgggccgcgagtactgggaaggcgtaatggccgtcaaggttgccgtgaacaaggaactggaagtgcagcgcgcggcgaaggccgttggcggcaacctgcaggccgaggtcacgctgtttgccgaggaaggcctgaccgctgacctggccaagctgagcaacgaactgcgcttcgtcctgatcacctcgaccgccagcctggcgccgtttgcccaggcgcctgcagatgccgttgcgaccgaagtcccgggtctcaagctcaaggtggtcaagtcctccttcgccaagtgcgcccgttgctggcactgccgtgaagatgtcggcgtgaaccccgagcatccggaaatctgcggtcgttgcgtcgacaacatctctggcgctggcgaggttcgtcactatgcctga

SEQ ID NO: 4 - DNA, 1785 bp, Pseudomonas sp.
  source 1..1785: mol_type = genomic DNA
atgactacaggcagtgctctgtacatcccgccttataaggcagacgaccaggatgtggtcgtcgaactcaacaaccgttttggccctgacgccttcaccgcccaggccacacgcaccggcatgccggtgctgtgggtggcgcgtgccaggctcgtcgaagtcctgagcttcctgcgcaacctgcccaagccgtacgtcatgctctatgacctgcatggcgtggacgagcgcctgcgcaccaaacgccaggggctgccgagcggcgccgacttcaccgtgttttaccacttgatgtcgcttgaacgtaacagcgacgtgatgatcaaggtggccctctccgagagcgacctgagcgttccgaccgtgaccggtatctggcccaacgccaactggtacgagcgtgaagtgtgggacatgttcggcatcgatttcccgggccacccgcacctgtcgcgcatcatgatgccgccgacctgggaaggtcacccgctgcgcaaggacttccctgcccgcgccaccgaattcgatccattcagcctgaccctcgccaagcaacagcttgaagaagaggctgcgcgcttccgtccggaagactggggcatgaagcgctccggcaccaacgaggactacatgttcctcaacctgggcccgaaccacccttcggctcacggtgccttccgcatcatcctgcaactggacggcgaagaaatcgtcgactgcgtaccggacatcggctaccaccaccgtggtgccgagaagatggccgagcgtcagtcgtggcacagcttcatcccgtacaccgaccgtatcgactacctcggcggcgtgatgaacaacctgccgtacgtgctctcggtcgagaagctggccggcatcaaggtgccggacaaggtcgacaccatccgcatcatgatggccgagttcttccggatcaccagccacctgctgttcctgggtacctatatccaggacgtcggcgccatgaccccggtgttcttcaccttcaccgaccgtcagcgtgcgtacaaggtcatcgaagccatcaccggtttccgcctgcaccccgcctggtaccgcatcggcggcgtcgcccacgacctgccacgtggctgggaacgcctggtcaaggaattcatcgactggatgcccaagcgtctggacgaataccagaaagccgccctggacaacagcatcctcaagggccggaccatcggcgtcgcccagtacaacaccaaagaggccctggaatggggcgtcaccggtgctggcctgcgttccaccggttgcgatttcgacctgcgtaaagcacgcccgtactccggctaccagaacttcgaattcgaagtgccgctggcggccaacggcgatgcctacgaccgctgcatcgtgcgcgtcgaagaaatgcgccagagcctgaagatcatcgaacagtgcatgcgcaacatgccggaaggcccgtacaaggcggatcacccgctgaccacgccgccgccgaaagagcgcacgctgcaacacatcgaaaccctgatcacgcacttcctgcaagtttcgtggggcccggtaatgccggccaacgaatccttccagatgatcgaagcgaccaagggtatcaacagttattacctgacgagcgatggcggcaccatgagctaccgcacccggattcgtacgccaagcttcccgcacctgcagcagatcccttcggtgatcaaaggtgaaatggtcgcggacttgattgcgtacctgggtagtatcgatttcgttatggccgacgtggaccgctaa

SEQ ID NO: 5 - DNA, 1059 bp, Pseudomonas sp.
  source 1..1059: mol_type = genomic DNA
atggacgacaacaagaagaaagccttggctgcggccctgggtcagatcgaacgtcaattcggcaagggtgccgtaatgcgtatgggcgatcacgaccgtcaggcgatcccggctatctccactggctctctgggtctggacatcgcgctcggcattggcggcctgccaaaaggccgtatcgttgagatctacggtcctgaatcttccggtaaaaccaccctgaccctgtcggtgattgcccaggcacaaaaaatgggcgccacctgtgcgttcgtcgatgccgagcacgccctggatcctgaatacgccggcaagctgggcgtcaacgttgacgacctgctggtttcgcaaccggacaccggtgagcaagcgctggaaatcaccgacatgctggtgcgctccaacgccatcgacgtgatcgtggtcgactccgtggcagccctggtacccaaggctgaaatcgaaggcgaaatgggcgacatgcacgtgggccttcaagcccgcttgatgtcccaggccctgcgtaaaatcaccggtaacatcaagaacgccaactgcttggtgatcttcatcaaccagatccggatgaagatcggcgtgatgttcggcagcccggaaaccaccaccggtggtaacgcgctgaagttctacgcttcggtccgtctggacatccgtcgtactggcgcggtgaaggaaggtgacgaggttgttggtagcgaaacccgcgttaaagtcgtgaagaacaaggtggccccgccgttccgtcaggccgagttccagattctctacggcaagggtatctacctgaacggcgagatgatcgacctgggcgtgctgcacggtttcgtcgagaaatccggtgcctggtatgcctacaacggcagcaagatcggccagggcaaggccaactcggccaagttccttgcggataacccggatgttgccgctacgctcgagaagcagatccgcgacaagttgctgaccccggcacctgacgtcaaggctgccgccaaccgcgagccggttgaagaagtagaagaagtcgacactgacatctga

SEQ ID NO: 6 - DNA, 3924 bp, Pseudomonas sp.
  source 1..3924: mol_type = genomic DNA
gtgggcctgcatgcggccttcaaatccgttttcccgatcatcagctactccggcaatgctgcgctggagtacgtcggttatcgcctgggcgaaccggcatttgatgtcaaagaatgcgtgttgcgcggtgttacgtacgccgtacctttgcgggtaaaagtccgtctgatcatcttcgacaaagagtcgtcgaacaaagcgatcaaggacatcaaagagcaagaagtctacatgggtgaaatccccctgatgactgagaacggtaccttcgttatcaacggtaccgagcgcgttatcgtttcccagctgcaccgttccccgggcgtgttctttgaccacgaccgcggcaagacgcacagctccggcaagctcctgtactccgcgcggatcattccgtaccgcggttcgtggttggacttcgagttcgacccgaaagactgcgtgttcgtgcgtatcgaccgtcgtcgcaagctgccggcctcggtactgctgcgcgcgctcggctataccactgagcaagtgctggacgctttctacaccactaacgtattccacctgagcggcgaaaccctcagcctggaactggtgcctcagcgcctgcgtggtgagattgccgttctggacatccaggacgacaaaggcaaggtcatcgttgaagccggccgccgtattaccgcgcgccacatcaaccagatcgaaaaagccggtatcaagacgctggaagtgcctctggactacgtcctgggtcgcaccaccgccaaggtcatcgtgcacccggccaccggcgaaatcctggctgaatgcaacaccgagctgaacaccgagatcctggcgaaaatcgccaagtcccaggttgttcgcatcgagaccctgtacaccaatgacatcgactgcggcccgttcgtctccgacaccctgaagatcgactccaccagcaaccaattggaagcgctggtcgagatctatcgcatgatgcgtcctggcgagccaccaaccaaagacgctgccgagaccctgttcaacaacctgttcttcagccctgagcgctatgacctgtctgcggtcggccggatgaagttcaaccgtcgtatcggtcgtaccgagatcgaaggttcgggcgtgctgtgcaaggaagacatcgtcgcggtactgaagaccctggtcgacatccgtaacggcaaaggcatcgtcgatgacatcgaccacctgggtaaccgtcgtgttcgctgcgtaggcgaaatggccgagaaccagttccgcgttggcctggtacgtgttgagcgtgcggtcaaagagcgtctgtcgatggccgaaagcgaaggcctgatgccgcaagacctgatcaacgccaagccagtggctgcggcggtgaaagagttcttcggttccagccagctctcgcagttcatggaccagaacaacccgctctccgagatcacccacaagcgccgtgtttccgcactgggcccgggcggtctgactcgtgagcgtgcaggctttgaagttcgtgacgtacacccgactcactacggtcgtgtatgcccgattgaaacgccggaaggtccgaacatcggtctgatcaactccctggctgcctacgcgcgcaccaaccagtacggcttcctcgagagcccgtaccgcgtggtgaaagacgcactggtcaccgacgagatcgtgttcctgtccgccatcgaagaagctgatcacgtgatcgctcaggcttcggccacgatgaacgacaagaaaatgctgatcgacgagctggtagctgttcgtcacttgaacgagttcaccgtcaaggcgccggaagacgtcaccttgatggacgtatcgccgaagcaggtagtttcggttgcagcgtcgctgatcccgttcctggagcacgatgacgccaaccgtgcgttgatgggttccaacatgcagcgtcaagctgtaccgaccctgcgcgccgacaagccgctggtaggtaccggcatggagcgtaacgtagcccgtgactccggcgtttgcgtcgtggctcgtcgtggcggcgtgatcgactccgttgatgccagccgtatcgtggttcgtgttgccgatgacgaagttgaaactggcgaagccggtgtcgacatctacaacctgaccaagtacacccgctccaaccagaacacctgcatcaaccagcgtccgctggtgagcaagggtgatcgggttcagcgcagcgacatcatggcagacggtccgtccaccgatatgggtgagctggcactgggtcagaacatgcgcatcgcgttcatggcctggaacggttacaacttcgaagactccatctgcctgtccgagcgtgtggttcaggaagatcgcttcaccacgatccacattcaggaactgacctgtgtggcgcgtgacaccaagcttgggccagaggaaatcactgcagacatcccgaacgtgggtgaagctgcactgaacaagctggacgaagccggtatcgtttacgtaggtgctgaagttggcgcaggcgacattctggtaggtaaggtcactccgaaaggcgagacccaactgactccggaagagaagctgttgcgtgccatcttcggtgaaaaagccagcgacgttaaagacacttccctgcgtgtacctaccggtaccaagggcactgtcatcgacgtacaggtcttcacccgtgacggcgttgagcgtgatgctcgtgcactgtccatcgagaagacccaactcgacgagatccgcaaggatctgaacgaagagttccgtatcgttgaaggcgcgaccttcgaacgtctgcgttccgccctggtaggccacaaggctgaaggcggcgcaggtctgaagaaaggtcaggacatcaccgacgaagtactcgacggtcttgagcacgggcagtggttcaaactgcgcatggctgaagatgctctgaacgagcagctcgagaaggcccaggcctacatcgtcgatcgtcgccgtctgctggacgacaagttcgaagacaagaagcgcaaactgcagcagggcgatgacctggctccaggcgtgctgaaaatcgtcaaggtttacctggcaatccgtcgccgcatccagccgggcgacaagatggccggtcgtcacggtaacaaaggtgtggtctccgtgatcatgccggttgaagacatgccgcacgatgccaatggcaccccggtcgatgtcgtcctcaacccgttgggcgtaccttcgcgtatgaacgttggtcagatccttgaaacccacctgggcctcgcggccaaaggtctgggcgagaagatcaaccgtatgatcgaagagcagcgcaaggttgcagacctgcgcaagttcctgcacgagatctacaacgagatcggcggtcgcaacgaagagctggacaccttctccgaccaggaaatcctggatctggcgaagaacctgcgcggcggcgttccaatggctaccccggtgttcgacggtgccaaggaaagcgaaatcaaggccatgctgaaactggcagacctgccagaaagcggccagatgcagctgttcgacggccgtaccggcaacaagtttgagcgcccggttactgttggctacatgtacatgctgaagctgaaccacttggtagacgacaagatgcacgctcgttctaccggttcgtacagcctggttacccagcagccgctgggtggtaaggctcagttcggtggtcagcgtttcggggagatggaggtctgggcactggaagcatacggtgctgcttacactctgcaagaaatgctcacagtgaagtcggacgatgtgaatggtcggaccaagatgtacaaaaacatcgtggacggcgatcaccgtatggagccgggcatgcccgagtctttcaacgtgttgatcaaagaaattcgttccctcggcatcgatatcgatctggaaaccgaataa

SEQ ID NO: 7 - DNA, 1851 bp, Pseudomonas sp.
  source 1..1851: mol_type = genomic DNA
atgtccggaaaagcgcaacagcagtctcgtatcaaagagttgatcacccttggtcgtgagcagaagtatctgacttacgcagaggtcaacgaccacctgcctgaggatatttcagatccagagcaggtggaagacatcatccgcatgattaatgacatggggatccccgtacacgagagtgctccggatgcggacgcccttatgttggccgacgccgataccgacgaggcagccgctgaagaagcagccgccgcgttggcagcggtcgagaccgacatcggtcgcacgactgaccctgtgcgcatgtatatgcgcgaaatgggtacggtagagctgctgacacgtgaaggcgaaatcgaaatcgccaagcgtatcgaagagggcatccgtgaagtgatgggcgcaatcgcgcacttcccaggcacggttgaccacattctctccgagtacactcgcgtcaccaccgaaggtggccgcctgtccgacgtcctgagcggttatatcgacccggacgacggcattgcgccacctgcggctgaaattccgccaccgatcgatgccaaggctgcgaaagcagacgacgacactgacgacgacgaagctgaagccagcaccgatgacgaagaagaagtcgaaagcggtccggatccgatcatcgcagcccagcgctttggcgcggtctccgatcagatggaactggctcgcaaggccctgaagaagcacggtcgcggcagcaagcaggcaactgctgaattgctggccctggctgaactcttcatgccgatcaaactggtaccgaagcaattcgaaggcctggttgagcgggttcgtagcgcccttgagcgtctgcgtgcacaagagcgtgcgatcatgcagctctgtgtccgtgatgcgcgcatgccgcgtgccgacttcctgcgccagttcccgggcaacgaagtcgacgaaagctggaccgacgcactggccaaaggcaaaagcaaatacgctgaagccattggtcgcttccaggccgacatcgttcgttgccagcagaagctgactgcattgcagaccgaaaccggtctgacgattgccgagatcaaggacatcaaccgtcgcatgtcgatcggtgaggccaaggcccgccgcgcgaagaaagagatggttgaagcgaacttgcgtctggtgatctccatcgccaagaagtacaccaaccgtggcctgcaattcctcgatctgatccaggaaggcaacatcggcttgatgaaagcggtggacaagttcgaataccgtcgcggctacaagttctcgacttatgccacctggtggatccgtcaggcgatcactcgctcgatcgccgaccaggcccgcaccatccgtattccggtgcacatgatcgagacgatcaacaagctcaaccgtatttcccggcagatgttgcaggaaatgggtcgcgaaccgaccccggaagagttgggcgaacgcatggaaatgcctgaggataaaatccgcaaggtattgaagatcgctaaagagccgatctccatggaaaccccgatcggtgatgacgaagactcccatctgggtgacttcatcgaagactcgaccatgcagtcgccaatcgatgttgctaccgttgagagcctgaaagaagcgactcgcgacgtactgtccggcctcactgcccgtgaagccaaggtactgcgcatgcgcttcggcatcgacatgaataccgaccacacccttgaggaggttggtaagcagttcgacgttacccgtgagcggattcgtcagatcgaagccaaggcgctgcgcaagctgcgccacccgacgagaagcgagcatttgcgctccttcctcgacgagtga